(19) United States
(12) Patent Application Publication
(10) Pub. No.: US 2015/0328285 A1
(43) Pub. Date: Nov. 19, 2015

(54) METHODS AND COMPOSITIONS USING FGF-9 TO ENHANCE NEOVASCULARIZATION AND REGENERATION

(71) Applicant: UNIVERSITY OF CENTRAL FLORIDA RESEARCH FOUNDATION, INC., Orlando, FL (US)

(72) Inventor: Dinendar Singla, Orlando, FL (US)

(21) Appl. No.: 14/389,408

(22) PCT Filed: Mar. 9, 2013
(86) PCT No.: PCT/US13/30082

§ 371 Date: Sep. 30, 2014

Related U.S. Application Data
(60) Provisional application No. 61/618,280, filed on Mar. 30, 2012.

(51) Int. Cl. A61K 38/18; A61K 35/545; A61K 38/13; A61K 45/06

(52) U.S. Cl. CPC A61K 38/1825; A61K 45/06; A61K 35/545; A61K 38/13

(57) ABSTRACT

In an aspect, the invention relates to compositions and methods of enhancing neovascularization. In an aspect, the invention relates to compositions and methods for attenuating vascular apoptosis. In an aspect, the invention relates to compositions and methods for inhibiting vascular apoptosis. This abstract is intended as a scanning tool for purposes of searching in the particular art and is not intended to be limiting of the present invention.

### CROSS REFERENCE TO RELATED PATENT APPLICATIONS

This application claims priority U.S. Provisional Application No. 61/618,280 filed Mar. 30, 2012, which is herein incorporated by reference in its entirety, and is related to U.S. Provisional Application No. 61/617,985 filed Mar. 30, 2012, titled “Methods and Compositions using FGF-8 to Enhance Cardiac Regeneration and Attenuate Adverse Cardiac Remodeling,” by Dinendar Singla, which is herein incorporated by reference in its entirety.

### STATEMENT REGARDING FEDERALLY SPONSORED RESEARCH

Parts of this invention were made with government support under 5R01HL090646-04 awarded by the National Institutes of Heath. The United States government has certain rights in the invention.

### BACKGROUND

Myocardial infarction (MI) leads to heart failure implicated by complex mechanisms of cardiac myocyte cell death, hypertrophy and fibrosis. The common therapeutic preferences to inhibit further deterioration of MI leading to end stage heart failure are very restricted. While heart transplantation is required to manage end stage heart failure patients, it is not readily available. Moreover, the development, progression and pathogenesis of myocardial infarction (MI) in type II diabetes are complex and severe compared with age and sex matched non-diabetic patients.

Therefore, new cellular therapies to repair and regenerate injured myocardium as well as to prevent the progression of adverse cardiac remodeling are under diligent examination. Cell therapy has emerged as a potential therapeutic approach for repairing injured myocardium. Thus far, cell transplantation in the mouse, rat, and human infarcted hearts has been studied employing a wide variety of cell types such as skeletal myoblasts, embryonic stem (ES) cells, c-kit positive cardiac stem cells, CD45^{+ve} bone marrow stem cells, mesenchymal stem cells (MSC), and hematopoietic stem cells. Indeed, adult stem cells in clinical trials have furnished significant insights into cell transplantation; however, their effectiveness is still in question. In contrast, ES cells have never reached clinical trials due to ethical and additional concerns. Thus, the optimal cell types for transplantation in the injured myocardium has yet to be identified.

Despite advances in understanding the physiology and pathophysiology of cardiac dysfunction, including myocardial infarction, there is still a scarcity of compounds that are both potent, efficacious, and safe in enhancing neovascularization and regeneration of the heart following MI. These needs and other needs are satisfied by the present invention.

### SUMMARY

Disclosed herein is a method of enhancing neovascularization following cardiac dysfunction in a subject in need thereof, the method comprising (i) administering to the subject fibroblast growth factor-9 primed induced pluripotent stem cells; (ii) administering to the subject conditioned medium of fibroblast growth factor-9 primed induced pluripotent stem cells; (iii) administering to the subject fibroblast growth factor-9; and/or (iv) administering to the subject fibroblast growth factor-9 and fibroblast growth factor-8; and determining neovascularization in the heart.

Disclosed herein is a method of attenuating vascular apoptosis and/or apoptosis-related mechanisms following cardiac dysfunction in a subject in need thereof comprising (i) administering to the subject fibroblast growth factor-9 primed induced pluripotent stem cells; (ii) administering to the subject conditioned medium of fibroblast growth factor-9 primed induced pluripotent stem cells; (iii) administering to the subject fibroblast growth factor-9; and/or (iv) administering to the subject a composition comprising fibroblast growth factor-9 and fibroblast growth factor-8; and determining apoptosis and/or apoptosis related mechanisms in the heart.

Disclosed herein is a composition of enhancing neovascularization in a subject, the composition comprising (i) fibroblast growth factor-9 primed induced pluripotent stem cells, (ii) conditioned medium of fibroblast growth factor-9 primed induced pluripotent stem cells; (iii) administering to the subject fibroblast growth factor-9; and/or (iv) administering to the subject a composition comprising fibroblast growth factor-9 and fibroblast growth factor-8.

Disclosed herein is a composition of attenuating vascular apoptosis and/or apoptosis-related mechanisms in a subject, the composition comprising (i) fibroblast growth factor-9 primed induced pluripotent stem cells; (ii) conditioned medium of fibroblast growth factor-9 primed induced pluripotent stem cells; (iii) administering to the subject fibroblast growth factor-9; and/or (iv) administering to the subject a composition comprising fibroblast growth factor-9 and fibroblast growth factor-8.

Disclosed herein is a method of generating cardiac induced pluripotent stem cells, the method comprising (i) inserting one or more nucleic acid constructs capable of expressing stem-cell like factors into a cardiac cell type, wherein the stem-cell like factors comprise Oct3/4, KIf4, Sox2, c-Myc, and/or a combination thereof; and (ii) obtaining the cardiac induced pluripotent stem cells stably expressing the stem-cell like factors in the cardiac cell type.

Disclosed herein is a method of inhibiting vascular apoptosis and/or apoptosis-related mechanisms comprising administering conditioned medium from induced pluripotent stem cells, wherein the conditioned medium is administered with or without fibroblast growth factor-9.

Disclosed herein is a method of inhibiting vascular apoptosis and/or apoptosis-related mechanisms comprising administering fibroblast growth factor-9.

### BRIEF DESCRIPTION OF THE FIGURES

The accompanying figures, which are incorporated in and constitute a part of this specification, illustrate several aspects and together with the description serve to explain the principles of the invention.

FIGS. 1A-D shows indicia of untransfected H9c2 cells and indicia of stably transfected iPS cells.

FIGS. 2A-I shows that iPS cells generated from embryoid bodies differentiate into cardiac myocytes, smooth muscles cells, and endothelial cells.

FIG. 3A shows that FGF-9-treated EBs stained positively for markers specific for ECs, VSMCs, and iPS cells; FIG. 3B provides a quantitative analysis of ECs and VSMCs following iPS and FGF-9 treatment.

FIG. 4A shows that iPS cells transplanted following MI differentiated into ECs and VSMCs in diabetic and non-diabetic mice and demonstrated neovascularization; FIG. 4B provides a quantitative analysis of ECs (bottom panel) and VSMCs (top panel) following treatment with iPS cells.

FIG. 5A shows c-kit positive cell activation in neovascularization in VSMCs (actin) and ECs (CD31) in diabetic and non-diabetic mice; FIG. 5B provides a quantitative analysis of positive c-kit+VSMCs (top panel) and c-kit+ECs (bottom panel) following MI and treatment with iPS cells or FGF-9.

FIG. 6A shows caspase activity and cell death detection following treatment with glucose, conditioned medium, FGF-9, and a combination thereof.

FIGS. 7A-F show the effect of iPS cells or FGF-9 following MI on artery/capillary formation.

FIGS. 8A-B show the effects of treatment with iPS cells and iPS cells with FGF-9 on cell differentiation.

FIG. 9 provides a quantitative analysis of FGF-9 treatment on miR-126 expression following MI.

FIGS. 10A-B show that treatment with iPS cells or FGF-9 blunted remodeling and improved cardiac function following MI.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples included therein.

Recently identified induced pluripotent stem cells (iPS cells) possess tremendous potential to treat heart disease, but their basic mechanisms of neovascularization in diabetic infarcted myocardium are largely unknown. Moreover, no attention has been paid to the dynamic and concurrent processes underlying post-MI neovascularization (angiogenesis and vasculogenesis), or the interaction of these processes with transplanted fibroblast growth factor-9 (FGF-9) primed neovascular committed iPS cells in non-diabetic and diabetic mice. The role of transplanted FGF-9 primed differentiated iPS cells or their conditioned medium (CM) in the endogenous activation of c-kit^{+ve} and FLK1^{-ve} cardiac progenitor cells (CPCs) and c-kit⁺ Flk-1^{+ve} progenitor cells (c-kit⁺ FLK-1^{+ve}-PCs) has never been determined in non-diabetic and diabetic mice.

Using four (4) factors (Oct3/4, Sox2, Klf4, and c-Myc), iPS cells were generated from H9c2 cells (originally isolated from embryonic rat heart ventricular tissue). These cells, which are referred to herein as “4F-iPS cells” or “iPS cells,” have the potential to differentiate into cardiac myocytes in vitro and in vivo. Following myocardial infarction, transplanted cardiac iPS cells inhibited post-MI remodeling and demonstrated cardiac regeneration.

### A. COMPOSITIONS

1. Compositions for Enhancing Neovascularization

Disclosed herein are compositions for enhancing neovascularization. In an aspect, disclosed herein is a composition for enhancing neovascularization in a subject, the composition comprising (i) fibroblast growth factor-9 primed induced pluripotent stem cells; (ii) conditioned medium of fibroblast growth factor-9 primed induced pluripotent stem cells; (iii) fibroblast growth factor-9; and/or (iv) a composition comprising fibroblast growth factor-9 and fibroblast growth factor-8; and determining neovascularization in the heart. For example, a disclosed composition for enhancing neovascularization in a subject comprises any one of the following combinations of fibroblast growth factor-9 primed induced pluripotent stem cells (FGF-9 primed iPS cells); conditioned medium of fibroblast growth factor-9 primed induced pluripotent stem cells (CM of FGF-9 primed iPS cells); fibroblast growth factor-9 (FGF-9); and a composition comprising fibroblast growth factor-9 and fibroblast growth factor-8 (FGF-9 and FGF-8).

TABLE 1
Listing of Disclosed Compositions for Enhancing Neovascularization
Components for Disclosed Compositions
for Enhancing Neovascularization

| Comprising A | Comprising B | | Comprising D |
|---|---|---|---|
| (i.e., FGF-9 | (i.e., CM of FGF-9 | Comprising C | (i.e., FGF-9 & |
| primed iPS cells) | iPS cells) | (i.e., FGF-9) | FGF-8) |
| A | B | C | D |
| A + B | B + A | C + A | D + A |
| A + C | B + C | C + B | D + B |
| A + D | B + D | C + D | D + C |
| A + B + C | B + A + C | C + A + B | D + A + B |
| A + B + D | B + A + D | C + A + D | D + A + C |
| A + C + D | B + C + D | C + B + D | D + B + C |
| A + B + C + D | B + A + C + D | C + A + B + D | D + A + B + C |

Thus, as detailed in the above table, a disclosed composition for enhancing neovascularization comprises one or two or three or all four of the following: (A) fibroblast growth factor-9 primed induced pluripotent stem cells; (B) conditioned media of fibroblast growth factor-9 primed induced pluripotent stem cells; (C) fibroblast growth factor-9; and (D) a composition comprising fibroblast growth factor-9 and fibroblast growth factor-8.

In an aspect, a disclosed composition for enhancing neovascularization enhances cell engraftment. In an aspect, a disclosed composition enhances cell proliferation. In an aspect, a disclosed composition enhances cell differentiation. In an aspect, a disclosed composition enhances cardiac myocyte differentiation in iPS cells or any other source of stem cells in regenerative medicine including, but not limited to, mesenchymal stem cells, adipocyte stems cells, and any other types of stem cells known to be effective by those of skill in the art. In an aspect, a disclosed composition enhances c-kit positive cells or any other endogenous heart stem cells.

In an aspect, a disclosed composition enhances cell proliferation, cell differentiation, and/or cell engraftment. For example, in an aspect, a disclosed composition enhances both cell engraftment and cell proliferation. In an aspect, a disclosed composition enhances both cell engraftment and cell differentiation. In an aspect, a disclosed composition enhances both cell proliferation and cell differentiation. In an aspect, a disclosed composition enhances cell engraftment, cell proliferation, and cell differentiation.

In an aspect, a disclosed composition for enhancing neovascularization enhances angiogenesis and/or vasculogenesis in the subject. For example, in an aspect, a disclosed composition enhances angiogenesis in the subject. In a further aspect, a disclosed composition enhances vasculogenesis in the subject. In an aspect, a disclosed composition enhances both angiogenesis and vasculogenesis in the subject.

In an aspect, a disclosed composition for enhancing neovascularization enhances cardiac function in the subject. In an aspect, enhancing cardiac function comprises increasing cardiac blood flow. In a further aspect, enhancing cardiac function comprises increasing cardiac capillary density. In yet another aspect, enhancing cardiac function comprises both increasing cardiac blood flow and increasing cardiac capillary density.

In an aspect, a disclosed composition for enhancing neovascularization attenuates vascular apoptosis and/or apoptosis-related mechanisms. In an aspect, the apoptosis and/or apoptosis-related mechanisms occurs in the induced pluripotent stem cells. In an aspect, the apoptosis and/or apoptosis-related mechanisms occurs in the cardiac tissue of the subject. In an aspect, a disclosed composition attenuates apoptosis and/or apoptosis-related mechanisms occurs in the iPS cells. In an aspect, a disclosed composition attenuates apoptosis and/or apoptosis-related mechanisms occurs in the cardiac tissue of the subject. In an aspect, a disclosed composition attenuates apoptosis and/or apoptosis-related mechanisms in both iPS cells and the cardiac tissue of the subject.

In an aspect, a disclosed composition for enhancing neovascularization increases miR-126 expression, decreases SPRED1 expression, and/or decreases PIK3R2 expression. For example, a disclosed composition increases miR-126 expression and decreases SPRED1 expression. In an aspect, a disclosed composition increases miR-126 expression and decreases PIK3R2 expression. In an aspect, a disclosed composition decreases SPRED1 expression and decreases PIK3R2 expression. In an aspect, a disclosed composition increases miR-126 expression, decreases SPRED1 expression, and decreases PIK3R2 expression.

In an aspect, the conditioned media of a disclosed composition for enhancing neovascularization comprises one or more anti-apoptotic and anti-fibrotic factors. In a further aspect, the one or more anti-apoptotic and anti-fibrotic factors comprise fibroblast growth factor-8 (FGF-8), fibroblast growth factor-9 (FGF-9), interleukin-10 (IL-10), and tissue inhibitor of matrix metalloproteinase-1 (TIMP-1).

In an aspect, the iPS cells differentiate into endothelial cells and/or vascular smooth muscle cells. In an aspect, the iPS cells differentiate into endothelial cells. In an aspect, the iPS cells differentiate into vascular smooth muscle cells. In an aspect, the iPS cells differentiate into endothelial cells and into vascular smooth muscle cells. In an aspect, the iPS cells are cardiac-committed. In an aspect, a disclosed composition enhances cardiac myocyte differentiation in iPS cells or any other source of stem cells in regenerative medicine including, but not limited to, mesenchymal stem cells, adipocyte stems cells, and any other types of stem cells known to be effective by those of skill in the art. In an aspect, a disclosed composition enhances c-kit positive cells or any other endogenous heart stem cells.

In an aspect, the subject has experienced cardiac dysfunction. In an aspect, the cardiac dysfunction is a cardiac ischemia/reperfusion event. In an aspect, the ischemia/reperfusion event is myocardial infarction, myocardial ischemia, myocardial reperfusion, subendocardial ischemia, Takayasu's arteritis, atrial fibrillation, hemorrhagic stroke, an event that occurs during cardiac surgery where a heart lung machine is used such as coronary artery bypass, or an event that occurs during the preservation of an organ for transplant. In an aspect, the subject has a congenital heart defect. In an aspect, the congenital heart defect is hypoplasia or pentalogy of Cantrell.

In an aspect, a disclosed composition for enhancing neovascularization in a subject is administered to the subject prior to cardiac dysfunction. In an aspect, a disclosed composition is administered to the subject during cardiac dysfunction. In an aspect, a disclosed composition is administered to the subject following cardiac dysfunction. For example, in an aspect, a disclosed composition is administered to the subject within 10, 15, 20, 25, 30, or more minutes following cardiac dysfunction. In an aspect, a composition is administered within 1, 2, 6, 12, 18, 24, or more hour following cardiac dysfunction.

In an aspect, a disclosed composition for enhancing neovascularization in a subject is administered to the subject one or more times. For example, in an aspect, a disclosed composition is administered to the subject prior to and during cardiac dysfunction. In an aspect, a disclosed composition is administered to the subject during and following cardiac dysfunction. For example, in an aspect, a disclosed composition is administered to the subject prior to and following cardiac dysfunction. In an aspect, a disclosed composition is administered to the subject prior to, during, and following cardiac dysfunction.

In an aspect, a disclosed composition for enhancing neovascularization in a subject comprises between 5,000 and 500,000 induced pluripotent stem cells (iPS cells). In an aspect, a disclosed composition comprises approximately 100,000 iPS cells. In an aspect, a disclosed composition comprises less than 100,000 iPS cells. In an aspect, a disclosed composition comprises more than 100,000 iPS cells.

In an aspect, a disclosed composition for enhancing neovascularization in a subject is administered to the subject in one intramyocardial injection. In an aspect, a disclosed composition is administered in two or more intramyocardial injections.

In an aspect, a disclosed composition for enhancing neovascularization in a subject in a subject is administered into a peri-infarct zone of the injured myocardium. In an aspect, disclosed composition is administered into an infarcted zone of the injured myocardium. In an aspect, a disclosed composition is administered into both a peri-infarct zone of the injured myocardium and an infarcted zone of the injured myocardium.

In an aspect, a disclosed composition for enhancing neovascularization in a subject comprises one or more immunosuppressive drugs Immunosuppressive drugs are known in the art. In an aspect, the one or more immunosuppressive drugs comprise corticosteroids, calcineurin inhibitors, anti-proliferatives, and mTOR inhibitors. In an aspect, the one or more immunosuppressive drugs can be a combination of immunosuppressive drugs. In an aspect, the one or more immunosuppressive drugs is cyclosporine A.

In an aspect, the induced pluripotent stem cells (iPS cells) of a disclosed composition are obtained from an autologous source. In an aspect, the iPS cells are obtained from an allogeneic source. In an aspect, the iPS cells are obtained from a syngeneic source. In an aspect, the iPS cells are obtained from a combination of sources.

In an aspect, the induced pluripotent stem cells of a disclosed composition for enhancing neovascularization in a subject are obtained from fibroblast cells. In a further aspect, the induced pluripotent stem cells are obtained from H9c2 cells. In an aspect, the fibroblast cells or the H9c2 cells are transfected with a vector comprising a nucleic acid molecule encoding at least one stemness factor. In an aspect, the at least one stemness factor comprises c-myc, oct 3/4, Klf4, nanog, or Sox2, or a combination thereof. For example, in an aspect, the stemness factors are c-myc, oct 3/4, Klf4, and Sox2.

In an aspect, a disclosed composition for enhancing neovascularization inhibits fibrosis and/or fibrosis-related mechanisms.

In an aspect, a disclosed composition for enhancing neovascularization protects myocardium including, but not limited to human myocardium, with or without the administration of iPS cells.

In an aspect, the subject is a mammal. In an aspect, the mammal is a primate. In an aspect, the mammal is a human. In an aspect, the human is a patient. In an aspect, the subject is diabetic.

2. Compositions for Attenuating Vascular Apoptosis

Disclosed herein is a composition for attenuating vascular apoptosis and/or apoptosis-related mechanisms in a subject, the composition comprising (i) fibroblast growth factor-9 primed induced pluripotent stem cells; (ii) conditioned medium of fibroblast growth factor-9 primed induced pluripotent stem cells; (iii) fibroblast growth factor-9; and/or (iv) a composition comprising fibroblast growth factor-9 and fibroblast growth factor-8. For example, a disclosed composition for enhancing neovascularization in a subject comprises any one of the following combinations of fibroblast growth factor-9 primed induced pluripotent stem cells (FGF-9 primed iPS cells); conditioned medium of fibroblast growth factor-9 primed induced pluripotent stem cells (CM of FGF-9 primed iPS cells); fibroblast growth factor-9 (FGF-9); and a composition comprising fibroblast growth factor-9 and fibroblast growth factor-8 (FGF-9 and FGF-8).

TABLE 2
Listing of Disclosed Compositions for Attenuating Vascular Apoptosis
Components for Disclosed Compositions
for Attenuating Vascular Apoptosis

| Comprising A | Comprising B | | Comprising D |
|---|---|---|---|
| (i.e., FGF-9 | (i.e., CM of FGF-9 | Comprising C | (i.e., FGF-9 & |
| primed iPS cells) | iPS cells) | (i.e., FGF-9) | FGF-8) |
| A | B | C | D |
| A + B | B + A | C + A | D + A |
| A + C | B + C | C + B | D + B |
| A + D | B + D | C + D | D + C |
| A + B + C | B + A + C | C + A + B | D + A + B |
| A + B + D | B + A + D | C + A + D | D + A + C |
| A + C + D | B + C + D | C + B + D | D + B + C |
| A + B + C + D | B + A + C + D | C + A + B + D | D + A + B + C |

Thus, as detailed in the above table, a disclosed composition for attenuating vascular apoptosis comprises one or two or three or all four of the following: (A) fibroblast growth factor-9 primed induced pluripotent stem cells; (B) conditioned media of fibroblast growth factor-9 primed induced pluripotent stem cells; (C) fibroblast growth factor-9; and (D) a composition comprising fibroblast growth factor-9 and fibroblast growth factor-8.

In an aspect, a disclosed composition for attenuating vascular apoptosis and/or apoptosis-related mechanisms enhances angiogenesis and/or vasculogenesis in the subject. For example, in an aspect, a disclosed composition enhances angiogenesis in the subject. In a further aspect, a disclosed composition enhances vasculogenesis in the subject. In an aspect, a disclosed composition enhances both angiogenesis and vasculogenesis in the subject.

In an aspect, a disclosed composition for attenuating vascular apoptosis and/or apoptosis-related mechanisms further enhances cardiac function in the subject. In an aspect, enhancing cardiac function comprises increasing cardiac blood flow. In a further aspect, enhancing cardiac function comprises increasing cardiac capillary density. In yet another aspect, enhancing cardiac function comprises both increasing cardiac blood flow and increasing cardiac capillary density.

In an aspect, the apoptosis and/or apoptosis-related mechanisms occurs in the induced pluripotent stem cells. In an aspect, the apoptosis and/or apoptosis-related mechanisms occurs in the cardiac tissue of the subject. In an aspect, a disclosed composition attenuates apoptosis and/or apoptosis-related mechanisms in the iPS cells. In an aspect, a disclosed composition attenuates apoptosis and/or apoptosis-related mechanisms in the cardiac tissue of the subject. In an aspect, a disclosed composition attenuates apoptosis and/or apoptosis-related mechanisms in the iPS cells and the cardiac tissue of the subject.

In an aspect, a disclosed composition for attenuating vascular apoptosis and/or apoptosis-related mechanisms increases miR-126 expression, decreases SPRED1 expression, and/or a decreases PIK3R2 expression. For example, a disclosed composition increases miR-126 expression and decreases SPRED1 expression. In an aspect, a disclosed composition increases miR-126 expression and decreases PIK3R2 expression. In an aspect, a disclosed composition decreases SPRED1 expression and decreases PIK3R2 expression. In an aspect, a disclosed composition increases miR-126 expression, decreases SPRED1 expression, and decreases PIK3R2 expression.

In an aspect, the conditioned media of a disclosed composition for attenuating vascular apoptosis and/or apoptosis-related mechanisms comprises one or more anti-apoptotic and anti-fibrotic factors. In a further aspect, the one or more anti-apoptotic and anti-fibrotic factors comprise fibroblast growth factor-8 (FGF-8), fibroblast growth factor-9 (FGF-9), interleukin-10 (IL-10), and tissue inhibitor of matrix metalloproteinase-1 (TIMP-1).

In an aspect, the iPS cells differentiate into endothelial cells and/or vascular smooth muscle cells. In an aspect, the iPS cells differentiate into endothelial cells. In an aspect, the iPS cells differentiate into vascular smooth muscle cells. In an aspect, the iPS cells differentiate into endothelial cells and vascular smooth muscle cells. In an aspect, the iPS cells are cardiac-committed. In an aspect, a disclosed composition enhances cardiac myocyte differentiation in iPS cells or any other source of stem cells in regenerative medicine including, but not limited to, mesenchymal stem cells, adipocyte stems cells, and any other types of stem cells known to be effective by those of skill in the art. In an aspect, a disclosed composition enhances c-kit positive cells or any other endogenous heart stem cells.

In an aspect, the subject has experienced cardiac dysfunction. In an aspect, the cardiac dysfunction is a cardiac ischemia/reperfusion event. In an aspect, the ischemia/reperfusion event is myocardial infarction, myocardial ischemia, myocardial reperfusion, subendocardial ischemia, Takayasu's arteritis, atrial fibrillation, hemorrhagic stroke, an event that occurs during cardiac surgery where a heart lung machine is used such as coronary artery bypass, or an event that occurs during the preservation of an organ for transplant. In an aspect, the subject has a congenital heart defect. In an aspect, the congenital heart defect is hypoplasia or pentalogy of Cantrell.

In an aspect, a disclosed composition for attenuating vascular apoptosis and/or apoptosis-related mechanisms in a subject is administered to the subject prior to cardiac dysfunction. In an aspect, a disclosed composition is administered to the subject during cardiac dysfunction. In an aspect, a disclosed composition is administered to the subject following cardiac dysfunction. For example, in an aspect, a disclosed composition is administered to the subject within 10, 15, 20, 25, 30, or more minutes following cardiac dysfunction. In an aspect, a composition is administered within 1, 2, 6, 12, 18, 24, or more hour following cardiac dysfunction.

In an aspect, a disclosed composition for attenuating vascular apoptosis and/or apoptosis-related mechanisms in a subject is administered to the subject one or more times. For example, in an aspect, a disclosed composition is administered to the subject prior to and during cardiac dysfunction. In an aspect, a disclosed composition is administered to the subject during and following cardiac dysfunction. For example, in an aspect, a disclosed composition is administered to the subject prior to and following cardiac dysfunction. In an aspect, a disclosed composition is administered to the subject prior to, during, and following cardiac dysfunction.

In an aspect, a disclosed composition for attenuating vascular apoptosis and/or apoptosis-related mechanisms in a subject comprises between 5,000 and 500,000 induced pluripotent stem cells (iPS cells). In an aspect, a composition comprises approximately 100,000 iPS cells. In an aspect, a composition comprises less than 100,000 iPS cells. In an aspect, a composition comprises more than 100,000 iPS cells.

In an aspect, a disclosed composition for attenuating vascular apoptosis and/or apoptosis-related mechanisms in a subject is administered to the subject in one intramyocardial injection. In an aspect, a disclosed composition is administered in two or more intramyocardial injections.

In an aspect, a disclosed composition for attenuating vascular apoptosis and/or apoptosis-related mechanisms in a subject is administered into a peri-infarct zone of the injured myocardium. In an aspect, disclosed composition is administered into an infarcted zone of the injured myocardium. In an aspect, a disclosed composition is administered into both a peri-infarct zone of the injured myocardium and an infarcted zone of the injured myocardium.

In an aspect, a disclosed composition for attenuating vascular apoptosis and/or apoptosis-related mechanisms in a subject comprises one or more immunosuppressive drugs. Immunosuppressive drugs are known in the art. In an aspect, the one or more immunosuppressive drugs comprise corticosteroids, calcineurin inhibitors, anti-proliferatives, and mTOR inhibitors. In an aspect, the one or more immunosuppressive drugs can be a combination of immunosuppressive drugs. In an aspect, the one or more immunosuppressive drugs is cyclosporine A.

In an aspect, the induced pluripotent stem cells (iPS cells) of a disclosed composition for attenuating vascular apoptosis and/or apoptosis-related mechanisms in a subject are obtained from an autologous source. In an aspect, the iPS cells are obtained from an allogeneic source. In an aspect, the iPS cells are obtained from a syngeneic source. In an aspect, the iPS cells are obtained from a combination of sources.

In an aspect, the induced pluripotent stem cells of a disclosed composition for attenuating vascular apoptosis and/or apoptosis-related mechanisms in a subject are obtained from fibroblast cells. In a further aspect, the induced pluripotent stem cells are obtained from H9c2 cells. In an aspect, the fibroblast cells or the H9c2 cells are transfected with a vector comprising a nucleic acid molecule encoding at least one stemness factor. In an aspect, the at least one stemness factor comprises c-myc, oct 3/4, Klf4, nanog, or Sox2, or a combination thereof. For example, in an aspect, the stemness factors are c-myc, oct 3/4, Klf4, and Sox2.

In an aspect, a disclosed composition for attenuating vascular apoptosis and/or apoptosis-related mechanisms inhibits fibrosis and/or fibrosis-related mechanisms.

In an aspect, a disclosed composition for attenuating vascular apoptosis and/or apoptosis-related mechanisms protects myocardium including, but not limited to human myocardium, with or without the administration of iPS cells.

In an aspect, the subject is a mammal. In an aspect, the mammal is a primate. In an aspect, the mammal is a human. In an aspect, the human is a patient. In an aspect, the subject is diabetic.

3. Pharmaceutical Compositions

In an aspect, the invention relates to pharmaceutical compositions comprising a disclosed composition for enhancing neovascularization. In an aspect, the invention relates to pharmaceutical compositions comprising a disclosed compositions for attenuating vascular apoptosis and/or apoptosis-related mechanisms. That is, a pharmaceutical composition can be provided comprising a therapeutically effective amount of at least one disclosed compositions and a pharmaceutically acceptable carrier.

### B. METHODS COMPRISING A DISCLOSED COMPOSITION

1. Enhancing Neovascularization

Disclosed herein are methods of enhancing neovascularization. In an aspect, disclosed herein is a method of enhancing neovascularization following cardiac dysfunction in a subject, the method comprising (i) administering to the subject fibroblast growth factor-9 primed induced pluripotent stem cells; (ii) administering to the subject conditioned medium of fibroblast growth factor-9 primed induced pluripotent stem cells; (iii) administering to the subject fibroblast growth factor-9; and/or (iv) administering to the subject a composition comprising fibroblast growth factor-9 and fibroblast growth factor-8. For example, in an aspect, a disclosed method of enhancing neovascularization in a subject comprises administering to the subject any one of the following combinations of fibroblast growth factor-9 primed induced pluripotent stem cells (FGF-9 primed iPS cells); conditioned medium of fibroblast growth factor-9 primed induced pluripotent stem cells (CM of FGF-9 primed iPS cells); fibroblast growth factor-9 (FGF-9); and a composition comprising fibroblast growth factor-9 and fibroblast growth factor-8 (FGF-9 and FGF-8).

TABLE 3
Listing of Disclosed Compositions for Methods of Enhancing
Neovascularization
Components for Disclosed Compositions for Methods
of Enhancing Neovascularization

| Comprising A | Comprising B | | Comprising D |
|---|---|---|---|
| (i.e., FGF-9 | (i.e., CM of FGF-9 | Comprising C | (i.e., FGF-9 & |
| primed iPS cells) | iPS cells) | (i.e., FGF-9) | FGF-8) |
| A | B | C | D |
| A + B | B + A | C + A | D + A |
| A + C | B + C | C + B | D + B |
| A + D | B + D | C + D | D + C |
| A + B + C | B + A + C | C + A + B | D + A + B |
| A + B + D | B + A + D | C + A + D | D + A + C |
| A + C + D | B + C + D | C + B + D | D + B + C |
| A + B + C + D | B + A + C + D | C + A + B + D | D + A + B + C |

Thus, as detailed in the above table, a disclosed method comprises administering one or two or three or all four of the following: (A) fibroblast growth factor-9 primed induced pluripotent stem cells; (B) conditioned media of fibroblast growth factor-9 primed induced pluripotent stem cells; (C) fibroblast growth factor-9; and (D) a composition comprising fibroblast growth factor-9 and fibroblast growth factor-8.

In an aspect, a disclosed method of enhancing neovascularization comprises enhancing cell engraftment. In an aspect, a disclosed method comprises enhancing cell proliferation. In an aspect, a disclosed method comprises enhancing cell differentiation.

In an aspect, a disclosed method enhances cardiac myocyte differentiation in iPS cells or any other source of stem cells in regenerative medicine including, but not limited to, mesenchymal stem cells, adipocyte stems cells, and any other types of stem cells known to be effective by those of skill in the art. In an aspect, a disclosed composition enhances c-kit positive cells or any other endogenous heart stem cells.

In an aspect, a disclosed method enhances cell proliferation, cell differentiation, and/or cell engraftment. For example, in an aspect, a disclosed method enhances both cell engraftment and cell proliferation. In an aspect, a disclosed method enhances both cell engraftment and cell differentiation. In an aspect, a disclosed method both cell proliferation and cell differentiation. In an aspect, a disclosed method enhances cell engraftment, cell proliferation, and cell differentiation.

In an aspect, a disclosed method enhances cardiac myocyte differentiation in iPS cells or any other source of stem cells in regenerative medicine including, but not limited to, mesenchymal stem cells, adipocyte stems cells, and any other types of stem cells known to be effective by those of skill in the art. In an aspect, a disclosed composition enhances c-kit positive cells or any other endogenous heart stem cells.

In an aspect, a disclosed method of enhancing neovascularization comprises enhancing angiogenesis and/or vasculogenesis in the subject. For example, in an aspect, a disclosed method comprises enhancing angiogenesis in the subject. In a further aspect, a disclosed method comprises enhancing vasculogenesis in the subject. In an aspect, a disclosed method comprises both enhancing angiogenesis and vasculogenesis in the subject.

In an aspect, a disclosed method of enhancing neovascularization further comprises enhancing cardiac function in the subject. In an aspect, enhancing cardiac function comprises increasing cardiac blood flow. In a further aspect, enhancing cardiac function comprises increasing cardiac capillary density. In yet another aspect, enhancing cardiac function comprises both increasing cardiac blood flow and increasing cardiac capillary density.

In an aspect, a disclosed method of enhancing neovascularization comprises attenuating vascular apoptosis. In an aspect, the apoptosis occurs in the induced pluripotent stem cells (iPS cells). In an aspect, the apoptosis and/or apoptosis-related mechanisms occurs in the cardiac tissue of the subject. In an aspect, the apoptosis and/or apoptosis-related mechanisms occurs both in the iPS cells and cardiac tissue of the subject. In an aspect, a disclosed composition attenuates apoptosis and/or apoptosis-related mechanisms in the iPS cells. In an aspect, a disclosed composition attenuates apoptosis and/or apoptosis-related mechanisms in the cardiac tissue of the subject. In an aspect, a disclosed composition attenuates apoptosis and/or apoptosis-related mechanisms in the iPS cells and the cardiac tissue of the subject.

In an aspect, a disclosed method of enhancing neovascularization comprises increasing miR-126 expression, decreasing SPRED1 expression, and/or decreasing PIK3R2 expression. For example, a disclosed method increases miR-126 expression and decreases SPRED1 expression. In an aspect, a disclosed method increases miR-126 expression and decreases PIK3R2 expression. In an aspect, a disclosed method decreases SPRED1 expression and decreases PIK3R2 expression. In an aspect, a disclosed method increases miR-126 expression, decreases SPRED1 expression, and decreases PIK3R2 expression.

In an aspect, the conditioned media of a disclosed method of enhancing neovascularization comprises one or more anti-apoptotic and anti-fibrotic factors. In a further aspect, the one or more anti-apoptotic and anti-fibrotic factors comprise fibroblast growth factor-8 (FGF-8), fibroblast growth factor-9 (FGF-9), interleukin-10 (IL-10), and tissue inhibitor of matrix metalloproteinase-1 (TIMP-1).

In an aspect, the iPS cells of a disclosed method of enhancing neovascularization differentiate into endothelial cells and/or vascular smooth muscle cells. In an aspect, the iPS cells differentiate into endothelial cells. In an aspect, the iPS cells differentiate into vascular smooth muscle cells. In an aspect, the iPS cells differentiate into both vascular smooth muscle cells and endothelial cells. In an aspect, the iPS cells are cardiac-committed. In an aspect, a disclosed method enhances cardiac myocyte differentiation in iPS cells or any other source of stem cells in regenerative medicine including, but not limited to, mesenchymal stem cells, adipocyte stems cells, and any other types of stem cells known to be effective by those of skill in the art. In an aspect, a disclosed composition enhances c-kit positive cells or any other endogenous heart stem cells.

In an aspect, the subject has experienced cardiac dysfunction. In an aspect, the cardiac dysfunction is a cardiac ischemia/reperfusion event. In an aspect, the ischemia/reperfusion event is myocardial infarction, myocardial ischemia, myocardial reperfusion, subendocardial ischemia, Takayasu's arteritis, atrial fibrillation, hemorrhagic stroke, an event that occurs during cardiac surgery where a heart lung machine is used such as coronary artery bypass, or an event that occurs during the preservation of an organ for transplant. In an aspect, the subject has a congenital heart defect. In an aspect, the congenital heart defect is hypoplasia or pentalogy of Cantrell.

In an aspect of a disclosed method of enhancing neovascularization, fibroblast growth factor-9 (FGF-9) is administered to the subject prior to the administration of the induced pluripotent stem cells (iPS cells). In an aspect, FGF-9 is administered to the subject during the administration of the iPS cells. In an aspect, FGF-9 is administered to the subject following the administration of the iPS cells. In an aspect, the administration of FGF-9 is repeated. For example, in an aspect, FGF-9 is administered to the subject prior to and during the administration of the iPS cells. In an aspect, FGF-9 is administered to the subject prior to and following the administration of the iPS cells. In an aspect, FGF-9 is administered to the subject during and following the administration of the iPS cells.

In an aspect of a disclosed method of enhancing neovascularization, a disclosed composition comprising fibroblast growth factor-8 (FGF-8) and fibroblast growth factor-9 (FGF-9) is administered to the subject prior to the administration of the induced pluripotent stem cells (iPS cells). In an aspect, a disclosed composition comprising FGF-8 and FGF-9 is administered to the subject during the administration of the iPS cells. In an aspect, a disclosed composition comprising FGF-8 and FGF-9 is administered to the subject following the administration of the iPS cells. In an aspect, the administration of a disclosed composition comprising FGF-8 and FGF-9 is repeated. For example, in an aspect, a disclosed composition comprising FGF-8 and FGF-9 is administered to the subject prior to and during the administration of the iPS cells. In an aspect, a disclosed composition comprising FGF-8 and FGF-9 is administered to the subject prior to and following the administration of the iPS cells. In an aspect, a disclosed composition comprising FGF-8 and FGF-9 is administered to the subject during and following the administration of the iPS cells.

In an aspect of a disclosed method of enhancing neovascularization, the conditioned media is administered to the subject prior to the administration of the induced pluripotent stem cells (iPS cells). In an aspect, the conditioned media is administered to the subject during the administration of the iPS cells. In an aspect, the conditioned media is administered to the subject following the administration of the iPS cells. In an aspect, the administration of the conditioned media is repeated. For example, in an aspect, the conditioned media is administered to the subject prior to and during the administration of the iPS cells. In an aspect, the conditioned media is administered to the subject prior to and following the administration of the iPS cells. In an aspect, the conditioned media is administered to the subject during and following the administration of the iPS cells.

In an aspect, a disclosed method of enhancing neovascularization comprises administering iPS cells to the subject prior to cardiac dysfunction. In an aspect, a disclosed method comprises administering iPS cells to the subject during cardiac dysfunction. In an aspect, a disclosed method comprises administering iPS cells to the subject following cardiac dysfunction. For example, in an aspect, a disclosed method comprises administering iPS cells to the subject within 10, 15, 20, 25, 30, or more minutes following cardiac dysfunction. In an aspect, a disclosed method comprises administering iPS cells to the subject within 1, 2, 6, 12, 18, 24, or more hour following cardiac dysfunction.

In an aspect of a disclosed method of enhancing neovascularization, the administration of induced pluripotent stem cells to the subject is repeated. For example, in an aspect, a disclosed method comprises administering iPS cells to the subject prior to and during cardiac dysfunction. In an aspect, a disclosed method comprises administering iPS cells to the subject during and following cardiac dysfunction. For example, in an aspect, a disclosed method comprises administering iPS cells to the subject prior to and following cardiac dysfunction. In an aspect, a disclosed method comprises administering iPS cells to the subject prior to, during, and following cardiac dysfunction.

In an aspect, a disclosed method of enhancing neovascularization in a subject comprises administering between 5,000 and 500,000 induced pluripotent stem cells (iPS cells). In an aspect, a disclosed method comprises administering approximately 100,000 iPS cells. In an aspect, a disclosed method comprises administering less than 100,000 iPS cells. In an aspect, a disclosed method comprises administering more than 100,000 iPS cells.

In an aspect, a disclosed method of enhancing neovascularization comprises administering iPS cells to the subject in one intramyocardial injection. In an aspect, a disclosed method of enhancing neovascularization comprises administering iPS cells to the subject in two or more intramyocardial injections.

In an aspect, a disclosed method of enhancing neovascularization in a subject comprises administering the iPS cells into a peri-infarct zone of the injured myocardium. In an aspect, a disclosed method of enhancing neovascularization comprises administering the iPS cells into an infarcted zone of the injured myocardium. In an aspect, a disclosed method of enhancing neovascularization in a subject comprises administering iPS cells into both a peri-infarct zone of the injured myocardium and an infarcted zone of the injured myocardium.

In an aspect, a disclosed method of enhancing neovascularization in a subject comprises administering one or more immunosuppressive drugs Immunosuppressive drugs are known in the art. In an aspect, the one or more immunosuppressive drugs comprise corticosteroids, calcineurin inhibitors, anti-proliferatives, and mTOR inhibitors. In an aspect, the one or more immunosuppressive drugs can be a combination of immunosuppressive drugs. In an aspect, the one or more immunosuppressive drugs is cyclosporine A.

In an aspect, the one or more immunosuppressive drugs is administered to the subject prior to, during, and/or following the administration of the induced pluripotent stem cells (iPS cells). In an aspect, the one or more immunosuppressive drugs is administered to the subject prior to the administration of iPS cells. In an aspect, the one or more immunosuppressive drugs is administered to the subject during the administration of iPS cells. In an aspect, the one or more immunosuppressive drugs is administered to the subject following the administration of iPS cells. In an aspect, the administration of the one or more immunosuppressive drugs is repeated. In an aspect, the one or more immunosuppressive drugs is administered to the subject prior to and during the administration of the iPS cells. In an aspect, the one or more immunosuppressive drugs is administered to the subject during and following the administration of the iPS cells. In an aspect, the one or more immunosuppressive drugs is administered to the subject prior to and following the administration of the iPS cells.

In an aspect, the induced pluripotent stem cells (iPS cells) of a disclosed method are obtained from an autologous source. In an aspect, iPS cells are obtained from an allogeneic source. In an aspect, iPS cells are obtained from a syngeneic source. In an aspect, iPS cells are obtained from a combination of sources.

In an aspect, the iPS cells of a disclosed method of enhancing neovascularization in a subject are obtained from fibroblast cells. In a further aspect, the iPS cells are obtained from H9c2 cells. In an aspect, the fibroblast cells or the H9c2 cells are transfected with a vector comprising a nucleic acid molecule encoding at least one stemness factor. In an aspect, the at least one stemness factor comprises c-myc, oct 3/4, Klf4, nanog, or Sox2, or a combination thereof. For example, in an aspect, the stemness factors are c-myc, oct 3/4, Klf4, and Sox2.

In an aspect, a disclosed method for enhancing neovascularization inhibits fibrosis and/or fibrosis-related mechanisms.

In an aspect, a disclosed method for enhancing neovascularization protects myocardium including, but not limited to human myocardium, with or without the administration of iPS cells.

In an aspect, the subject is a mammal. In an aspect, the mammal is a primate. In an aspect, the mammal is a human. In an aspect, the human is a patient. In an aspect, the subject is diabetic.

2. Attenuating Vascular Apoptosis and/or Apoptosis-Related Mechanisms

Disclosed herein are methods of attenuating vascular apoptosis and/or apoptosis-related mechanisms. In an aspect, disclosed herein is a method of attenuating vascular apoptosis and/or apoptosis-related mechanisms following cardiac dysfunction in a subject in need thereof, the method comprising (i) administering to the subject fibroblast growth factor-9 primed induced pluripotent stem cells; (ii) administering to the subject conditioned medium of fibroblast growth factor-9 primed induced pluripotent stem cells; (iii) administering to the subject fibroblast growth factor-9; and/or (iv) administering to the subject a composition comprising fibroblast growth factor-9 and fibroblast growth factor-8. For example, in an aspect, a disclosed method of attenuating vascular apoptosis and/or apoptosis-related mechanisms comprises administering to the subject any one of the following combinations of fibroblast growth factor-9 primed induced pluripotent stem cells (FGF-9 primed iPS cells); conditioned medium of fibroblast growth factor-9 primed induced pluripotent stem cells (CM of FGF-9 primed iPS cells); fibroblast growth factor-9 (FGF-9); and a composition comprising fibroblast growth factor-9 and fibroblast growth factor-8 (FGF-9 and FGF-8).

TABLE 4
Listing of Disclosed Compositions for Methods of Attenuating
Vascular Apoptosis
Components for Disclosed Compositions for Methods
of Attenuating Vascular Apoptosis

| Comprising A | Comprising B | | Comprising D |
|---|---|---|---|
| (i.e., FGF-9 | (i.e., CM of FGF-9 | Comprising C | (i.e., FGF-9 & |
| primed iPS cells) | iPS cells) | (i.e., FGF-9) | FGF-8) |
| A | B | C | D |
| A + B | B + A | C + A | D + A |
| A + C | B + C | C + B | D + B |
| A + D | B + D | C + D | D + C |
| A + B + C | B + A + C | C + A + B | D + A + B |
| A + B + D | B + A + D | C + A + D | D + A + C |
| A + C + D | B + C + D | C + B + D | D + B + C |
| A + B + C + D | B + A + C + D | C + A + B + D | D + A + B + C |

Thus, as detailed in the above table, a disclosed method comprises administering one or two or three or all four of the following: (A) fibroblast growth factor-9 primed induced pluripotent stem cells; (B) conditioned media of fibroblast growth factor-9 primed induced pluripotent stem cells; (C) fibroblast growth factor-9; and (D) a composition comprising fibroblast growth factor-9 and fibroblast growth factor-8.

In an aspect, a disclosed method of attenuating vascular apoptosis and/or apoptosis-related mechanisms comprises enhancing angiogenesis and/or vasculogenesis in the subject. For example, in an aspect, a disclosed method comprises enhancing angiogenesis in the subject. In a further aspect, a disclosed method comprises enhancing vasculogenesis in the subject. In an aspect, a disclosed method comprises both enhancing angiogenesis and vasculogenesis in the subject.

In an aspect, a disclosed method of attenuating vascular apoptosis and/or apoptosis-related mechanisms comprises enhancing cardiac function in the subject. In an aspect, enhancing cardiac function comprises increasing cardiac blood flow. In a further aspect, enhancing cardiac function comprises increasing cardiac capillary density. In yet another aspect, enhancing cardiac function comprises increasing cardiac blood flow and increasing cardiac capillary density.

In an aspect, the apoptosis and/or apoptosis-related mechanisms occurs in the induced pluripotent stem cells (iPS cells). In an aspect, the apoptosis and/or apoptosis-related mechanisms occurs in the cardiac tissue of the subject. In an aspect, the apoptosis and/or apoptosis-related mechanisms occurs in both the iPS cells and the cardiac tissue of the subject. In an aspect, a disclosed composition attenuates apoptosis and/or apoptosis-related mechanisms in the iPS cells. In an aspect, a disclosed composition attenuates apoptosis and/or apoptosis-related mechanisms in the cardiac tissue of the subject. In an aspect, a disclosed composition attenuates apoptosis and/or apoptosis-related mechanisms in iPS cells and the cardiac tissue of the subject.

In an aspect, a disclosed method of attenuating vascular apoptosis and/or apoptosis-related mechanisms comprises increasing miR-126 expression, decreasing SPRED1 expression, and/or decreasing PIK3R2 expression. For example, a disclosed method increases miR-126 expression and decreases SPRED1 expression. In an aspect, a disclosed method increases miR-126 expression and decreases PIK3R2 expression. In an aspect, a disclosed method decreases SPRED1 expression and decreases PIK3R2 expression. In an aspect, a disclosed method increases miR-126 expression, decreases SPRED1 expression, and decreases PIK3R2 expression.

In an aspect, the conditioned media of a disclosed method of attenuating vascular apoptosis and/or apoptosis-related mechanisms comprises one or more anti-apoptotic and anti-fibrotic factors. In a further aspect, the one or more anti-apoptotic and anti-fibrotic factors comprise fibroblast growth factor-8 (FGF-8), fibroblast growth factor-9 (FGF-9), interleukin-10 (IL-10), and tissue inhibitor of matrix metalloproteinase-1 (TIMP-1).

In an aspect, the iPS cells of a disclosed method of attenuating vascular apoptosis and/or apoptosis-related mechanisms differentiate into endothelial cells and/or vascular smooth muscle cells. In an aspect, the iPS cells differentiate into endothelial cells. In an aspect, the iPS cells differentiate into vascular smooth muscle cells. In an aspect, the iPS cells differentiate into both vascular smooth muscle cells and endothelial cells. In an aspect, the iPS cells are cardiac-committed. In an aspect, a disclosed method enhances cardiac myocyte differentiation in iPS cells or any other source of stem cells in regenerative medicine including, but not limited to, mesenchymal stem cells, adipocyte stems cells, and any other types of stem cells known to be effective by those of skill in the art. In an aspect, a disclosed composition enhances c-kit positive cells or any other endogenous heart stem cells.

In an aspect, the subject has experienced cardiac dysfunction. In an aspect, the cardiac dysfunction is a cardiac ischemia/reperfusion event. In an aspect, the ischemia/reperfusion event is myocardial infarction, myocardial ischemia, myocardial reperfusion, subendocardial ischemia, Takayasu's arteritis, atrial fibrillation, hemorrhagic stroke, an event that occurs during cardiac surgery where a heart lung machine is used such as coronary artery bypass, or an event that occurs during the preservation of an organ for transplant. In an aspect, the subject has a congenital heart defect. In an aspect, the congenital heart defect is hypoplasia or pentalogy of Cantrell.

In an aspect of a disclosed method of attenuating vascular apoptosis and/or apoptosis-related mechanisms, fibroblast growth factor-9 (FGF-9) is administered to the subject prior to the administration of induced pluripotent stem cells (iPS cells). In an aspect, FGF-9 is administered to the subject during the administration of iPS cells. In an aspect, FGF-9 is administered to the subject following the administration of iPS cells. In an aspect, the administration of FGF-9 is repeated. For example, in an aspect, FGF-9 is administered to the subject prior to and during the administration of iPS cells. In an aspect, FGF-9 is administered to the subject prior to and following the administration of iPS cells. In an aspect, FGF-9 is administered to the subject during and following the administration of iPS cells.

In an aspect of a disclosed method of attenuating vascular apoptosis and/or apoptosis-related mechanisms, a disclosed composition comprising fibroblast growth factor-8 (FGF-8) and fibroblast growth factor-9 (FGF-9) is administered to the subject prior to the administration of the induced pluripotent stem cells (iPS cells). In an aspect, a disclosed composition comprising FGF-8 and FGF-9 is administered to the subject during the administration of iPS cells. In an aspect, a disclosed composition comprising FGF-8 and FGF-9 is administered to the subject following the administration of iPS cells. In an aspect, the administration of a disclosed composition comprising FGF-8 and FGF-9 is repeated. For example, in an aspect, a disclosed composition comprising FGF-8 and FGF-9 is administered to the subject prior to and during the administration of iPS cells. In an aspect, a disclosed composition comprising FGF-8 and FGF-9 is administered to the subject prior to and following the administration of iPS cells. In an aspect, a disclosed composition comprising FGF-8 and FGF-9 is administered to the subject during and following the administration of iPS cells.

In an aspect of a disclosed method of attenuating vascular apoptosis and/or apoptosis-related mechanisms, the conditioned media is administered to the subject prior to the administration of the induced pluripotent stem cells (iPS cells). In an aspect, the conditioned media is administered to the subject during the administration of iPS cells. In an aspect, the conditioned media is administered to the subject following the administration of iPS cells. In an aspect, the administration of the conditioned media is repeated. For example, in an aspect, the conditioned media administered to the subject prior to and during the administration of iPS cells. In an aspect, the conditioned media is administered to the subject prior to and following the administration of iPS cells. In an aspect, the conditioned media is administered to the subject during and following the administration of iPS cells.

In an aspect, a disclosed method of attenuating vascular apoptosis and/or apoptosis-related mechanisms comprises administering iPS cells to the subject prior to cardiac dysfunction. In an aspect, a disclosed method comprises administering iPS cells to the subject during cardiac dysfunction. In an aspect, a disclosed method comprises administering iPS cells to the subject following cardiac dysfunction. For example, in an aspect, a disclosed method comprises administering the iPS cells to the subject within 10, 15, 20, 25, 30, or more minutes following cardiac dysfunction. In an aspect, a disclosed method comprises administering the iPS cells to the subject within 1, 2, 6, 12, 18, 24, or more hour following cardiac dysfunction.

In an aspect of a disclosed method of attenuating vascular apoptosis and/or apoptosis-related mechanisms, the administration of induced pluripotent stem cells to the subject is repeated. For example, in an aspect, a disclosed method of attenuating vascular apoptosis and/or apoptosis-related mechanisms comprises administering iPS cells to the subject prior to and during cardiac dysfunction. In an aspect, a disclosed method comprises administering iPS cells to the subject during and following cardiac dysfunction. For example, in an aspect, a disclosed method comprises administering iPS cells to the subject prior to and following cardiac dysfunction. In an aspect, a disclosed method comprises administering iPS cells to the subject prior to, during, and following cardiac dysfunction.

In an aspect, a disclosed method of attenuating vascular apoptosis and/or apoptosis-related mechanisms in a subject comprises administering between 5,000 and 500,000 induced pluripotent stem cells (iPS cells). In an aspect, a disclosed method comprises administering approximately 100,000 iPS cells. In an aspect, a disclosed method comprises administering less than 100,000 iPS cells. In an aspect, a disclosed method comprises administering more than 100,000 iPS cells.

In an aspect, a disclosed method of attenuating vascular apoptosis and/or apoptosis-related mechanisms comprises administering the iPS cells to the subject in one intramyocardial injection. In an aspect, a disclosed method of attenuating vascular apoptosis and/or apoptosis-related mechanisms comprises administering iPS cells to the subject in two or more intramyocardial injections.

In an aspect, a disclosed method of attenuating vascular apoptosis and/or apoptosis-related mechanisms in a subject comprises administering iPS cells into a peri-infarct zone of the injured myocardium. In an aspect, disclosed method of attenuating vascular apoptosis and/or apoptosis-related mechanisms comprises administering the iPS cells into an infarcted zone of the injured myocardium. In an aspect, disclosed method comprises administering the iPS cells into both a peri-infarct zone of the injured myocardium and an infarcted zone of the injured myocardium.

In an aspect, a disclosed method of attenuating vascular apoptosis and/or apoptosis-related mechanisms in a subject comprises administering one or more immunosuppressive drugs. Immunosuppressive drugs are known in the art. In an aspect, the one or more immunosuppressive drugs comprise corticosteroids, calcineurin inhibitors, anti-proliferatives, and mTOR inhibitors. In an aspect, the one or more immunosuppressive drugs can be a combination of immunosuppressive drugs. In an aspect, the one or more immunosuppressive drugs is cyclosporine A.

In an aspect, the one or more immunosuppressive drugs is administered to the subject prior to, during, and/or following the administration of the induced pluripotent stem cells (iPS cells). In an aspect, the one or more immunosuppressive drugs is administered to the subject prior to the administration of iPS cells. In an aspect, the one or more immunosuppressive drugs is administered to the subject during the administration of iPS cells. In an aspect, the one or more immunosuppressive drugs is administered to the subject following the administration of iPS cells. In an aspect, the administration of the one or more immunosuppressive drugs is repeated. In an aspect, the one or more immunosuppressive drugs is administered to the subject prior to and during the administration of iPS cells. In an aspect, the one or more immunosuppressive drugs is administered to the subject during and following the administration of iPS cells. In an aspect, the one or more immunosuppressive drugs is administered to the subject prior to and following the administration of iPS cells.

In an aspect, the induced pluripotent stem cells (iPS cells) of a disclosed method of attenuating vascular apoptosis and/or apoptosis-related mechanisms are obtained from an autologous source. In an aspect, the iPS cells are obtained from an allogeneic source. In an aspect, the iPS cells are obtained from a syngeneic source. In an aspect, the iPS cells are obtained from a combination of sources.

In yet another aspect, the iPS cells of a disclosed method of attenuating vascular apoptosis and/or apoptosis-related mechanisms in a subject are obtained from fibroblast cells. In a further aspect, the iPS cells are obtained from H9c2 cells. In an aspect, the fibroblast cells or the H9c2 cells are transfected with a vector comprising a nucleic acid molecule encoding at least one stemness factor. In an aspect, the at least one stemness factor comprises c-myc, oct 3/4, Klf4, nanog, or Sox2, or a combination thereof. For example, in an aspect, the stemness factors are c-myc, oct 3/4, Klf4, and Sox2.

In an aspect, a disclosed method for attenuating vascular apoptosis and/or apoptosis-related mechanisms inhibits fibrosis and/or fibrosis-related mechanisms.

In an aspect, a disclosed method for attenuating vascular apoptosis and/or apoptosis-related mechanisms protects myocardium including, but not limited to human myocardium, with or without the administration of iPS cells.

In an aspect, the subject is a mammal. In an aspect, the mammal is a primate. In an aspect, the mammal is a human. In an aspect, the human is a patient. In an aspect, the subject is diabetic.

3. Generating Induced Pluripotent Stem Cells

Disclosed herein is a method of generating cardiac induced pluripotent stem cells (iPS cells). In an aspect, the method of generating cardiac induced pluripotent stem cells comprises (i) inserting one or more nucleic acid constructs capable of expressing stem-cell like factors into a cardiac cell type, and (ii) obtaining the cardiac induced pluripotent stem cells stably expressing the stem-cell like factors in the cardiac cell type. In an aspect, the stem-cell like factors comprise Oct3/4, KIf4, Sox2, nanog, and c-Myc, or a combination thereof. In an aspect, the iPS cells differentiate into cardiac myocytes. In a further aspect, the iPS cells are cardiac-committed.

In an aspect, a disclosed method enhances cardiac myocyte differentiation in iPS cells or any other source of stem cells in regenerative medicine including, but not limited to, mesenchymal stem cells, adipocyte stems cells, and any other types of stem cells known to be effective by those of skill in the art. In an aspect, a disclosed composition enhances c-kit positive cells or any other endogenous heart stem cells.

In an aspect, the iPS cells are obtained from an autologous source. In an aspect, the iPS cells are obtained from an allogeneic source. In an aspect, the iPS cells are obtained from a syngeneic source. In yet another aspect, the iPS cells are obtained from fibroblast cells. In an aspect, the iPS cells are obtained from a combination of sources. In a further aspect, the iPS cells are obtained from H9c2 cells. In an aspect, the fibroblast cells or the H9c2 cells are transfected with a vector comprising a nucleic acid molecule encoding at least one stemness factor.

In an aspect, the disclosed induced pluripotent stem cells are used in a method of enhancing revascularization in a subject. In an aspect, the disclosed induced pluripotent stem cells are used in a method of attenuating vascular apoptosis and/or apoptosis-related mechanisms in a subject. In an aspect, the subject is a mammal. In an aspect, the mammal is a primate. In an aspect, the mammal is a human. In an aspect, the human is a patient. In an aspect, the subject has diabetes. In an aspect, the subject has experienced cardiac dysfunction. In an aspect, the cardiac dysfunction is a cardiac ischemia/reperfusion event. In an aspect, the ischemia/reperfusion event is myocardial infarction, myocardial ischemia, myocardial reperfusion, subendocardial ischemia, Takayasu's arteritis, atrial fibrillation, hemorrhagic stroke, an event that occurs during cardiac surgery where a heart lung machine is used such as coronary artery bypass, or an event that occurs during the preservation of an organ for transplant. In an aspect, the subject has a congenital heart defect. In an aspect, the congenital heart defect is hypoplasia or pentalogy of Cantrell.

In an aspect, the disclosed iPS cells are primed with fibroblast growth factor-9. In an aspect, the iPS cells are administered with one or more of the following: conditioned media of fibroblast growth factor-9 primed induced pluripotent stem cells, fibroblast growth factor-9, and a disclosed composition comprising fibroblast growth factor-8 and fibroblast growth factor-9.

In an aspect, the disclosed induced pluripotent stem cells (iPS cells) improve angiogenesis in a subject. In an aspect, the disclosed iPS cells enhance cardiac function in a subject. In an aspect, the disclosed iPS cells improve angiogenesis and enhance cardiac function in the subject. In an aspect, enhancing cardiac function comprises one or more of (i) improving left ventricular function, (ii) improving fractional shortening, (iii) improving ejection fraction, (iv) reducing end-diastolic volume, (v) decreasing left ventricular mass, and/or (vi) normalizing of heart geometry.

In an aspect, iPS cells inhibit fibrosis and/or fibrosis-related mechanisms.

In an aspect, iPS cells protect myocardium including, but not limited to human myocardium, with or without the administration of iPS cells.

In an aspect, the disclosed induced pluripotent stem cells inhibit or decrease necrosis. In an aspect, the disclosed induced pluripotent stem cells inhibit or decrease apoptosis and/or apoptosis-related mechanisms.

4. Inhibiting Vascular Apoptosis and/or Apoptosis-Related Mechanisms

Disclosed herein is a method of inhibiting vascular apoptosis. In an aspect, the method of inhibiting vascular apoptosis comprises administering to a subject conditioned medium from induced pluripotent stem cells. In an aspect, a disclosed method of inhibiting vascular apoptosis comprises administering to a subject fibroblast growth factor-9. In an aspect, the subject is a mammal. In an aspect, the mammal is a primate. In an aspect, the mammal is a human. In an aspect, the human is a patient. In an aspect, the subject is diabetic.

In an aspect, the subject has experienced cardiac dysfunction. In an aspect, the cardiac dysfunction is a cardiac ischemia/reperfusion event. In an aspect, the ischemia/reperfusion event is myocardial infarction, myocardial ischemia, myocardial reperfusion, subendocardial ischemia, Takayasu's arteritis, atrial fibrillation, hemorrhagic stroke, an event that occurs during cardiac surgery where a heart lung machine is used such as coronary artery bypass, or an event that occurs during the preservation of an organ for transplant. In an aspect, the subject has a congenital heart defect. In an aspect, the congenital heart defect is hypoplasia or pentalogy of Cantrell.

In an aspect, a disclosed method of inhibiting vascular apoptosis and/or apoptosis-related mechanisms comprises enhancing angiogenesis and/or vasculogenesis in the subject. For example, in an aspect, a disclosed method comprises enhancing angiogenesis in the subject. In a further aspect, a disclosed method comprises enhancing vasculogenesis in the subject. In an aspect, a disclosed method comprises both enhancing angiogenesis and vasculogenesis in the subject.

In an aspect, a disclosed method of inhibiting vascular apoptosis and/or apoptosis-related mechanisms comprises enhancing cardiac function in the subject. In an aspect, enhancing cardiac function comprises increasing cardiac blood flow. In a further aspect, enhancing cardiac function comprises increasing cardiac capillary density. In yet another aspect, enhancing cardiac function comprises both increasing cardiac blood flow and increasing cardiac capillary density.

In an aspect, a disclosed method of inhibiting vascular apoptosis and/or apoptosis-related mechanisms further comprises increasing miR-126 expression, decreasing SPRED1 expression, decreasing PIK3R2 expression, and/or a combination thereof. For example, a disclosed method increases miR-126 expression and decreases SPRED1 expression. In an aspect, a disclosed method increases miR-126 expression and decreases PIK3R2 expression. In an aspect, a disclosed method decreases SPRED1 expression and decreases PIK3R2 expression. In an aspect, a disclosed method increases miR-126 expression, decreases SPRED1 expression, and decreases PIK3R2 expression.

In an aspect, the conditioned media of a disclosed method of inhibiting vascular apoptosis and/or apoptosis-related mechanisms comprises one or more anti-apoptotic and anti-fibrotic factors. In a further aspect, the one or more anti-apoptotic and anti-fibrotic factors comprise fibroblast growth factor-8 (FGF-8), fibroblast growth factor-9 (FGF-9), interleukin-10 (IL-10), and tissue inhibitor of matrix metalloproteinase-1 (TIMP-1).

In an aspect, a disclosed method of inhibiting vascular apoptosis and/or apoptosis-related mechanisms further comprises administering one or more of the following: (i) fibroblast growth factor-9 primed induced pluripotent stem cells; (ii) conditioned medium of fibroblast growth factor-9 primed induced pluripotent stem cells FGF-9 primed iPS cells); (iii) fibroblast growth factor-9; and/or (iv) a composition comprising fibroblast growth factor-9 and fibroblast growth factor-8.

For example, in an aspect, a disclosed method of inhibiting vascular apoptosis and/or apoptosis-related mechanisms comprising administering to the subject conditioned medium from induced pluripotent stem cells further comprises administering (i) fibroblast growth factor-9 primed induced pluripotent stem cells (FGF-9 primed iPS cells); (ii) fibroblast growth factor-9; and/or (iii) a composition comprising fibroblast growth factor-9 and fibroblast growth factor-8 (FGF-9 and FGF-8).

For example, in an aspect, a disclosed method of inhibiting vascular apoptosis and/or apoptosis-related mechanisms comprising administering to the subject FGF-9 can further comprises administering (i) fibroblast growth factor-9 primed induced pluripotent stem cells (FGF-9 primed iPS cells); (ii) conditioned medium of fibroblast growth factor-9 primed induced pluripotent stem cells; and/or (iii) a composition comprising fibroblast growth factor-9 and fibroblast growth factor-8 (FGF-9 and FGF-8).

Thus, as detailed below, a disclosed method of inhibiting vascular apoptosis and/or apoptosis-related mechanisms comprises any combination (such as one or two or three or all four) of the following components: (A) fibroblast growth factor-9 primed induced pluripotent stem cells; (B) conditioned media of fibroblast growth factor-9 primed induced pluripotent stem cells; (C) fibroblast growth factor-9; and (D) a composition comprising fibroblast growth factor-9 and fibroblast growth factor-8.

TABLE 5
Listing of Disclosed Compositions for Methods of Inhibiting
Vascular Apoptosis
Components for Disclosed Compositions for Methods
of Inhibiting Vascular Apoptosis

| Comprising A | Comprising B | | Comprising D |
|---|---|---|---|
| (i.e., FGF-9 | (i.e., CM of FGF-9 | Comprising C | (i.e., FGF-9 & |
| primed iPS cells) | iPS cells) | (i.e., FGF-9) | FGF-8) |
| A | B | C | D |
| A + B | B + A | C + A | D + A |
| A + C | B + C | C + B | D + B |
| A + D | B + D | C + D | D + C |
| A + B + C | B + A + C | C + A + B | D + A + B |
| A + B + D | B + A + D | C + A + D | D + A + C |
| A + C + D | B + C + D | C + B + D | D + B + C |
| A + B + C + D | B + A + C + D | C + A + B + D | D + A + B + C |

In an aspect, the iPS cells of a disclosed method of inhibiting vascular apoptosis and/or apoptosis-related mechanisms differentiate into endothelial cells and/or vascular smooth muscle cells. In an aspect, the iPS cells differentiate into endothelial cells. In an aspect, the iPS cells differentiate into vascular smooth muscle cells. In an aspect, the iPS cells differentiate into both endothelial cells and vascular smooth muscle cells. In an aspect, the iPS cells are cardiac-committed. In an aspect, a disclosed method enhances cardiac myocyte differentiation in iPS cells or any other source of stem cells in regenerative medicine including, but not limited to, mesenchymal stem cells, adipocyte stems cells, and any other types of stem cells known to be effective by those of skill in the art. In an aspect, a disclosed composition enhances c-kit positive cells or any other endogenous heart stem cells.

In an aspect, the induced pluripotent stem cells (iPS cells) of a disclosed method of inhibiting vascular apoptosis and/or apoptosis-related mechanisms are obtained from an autologous source. In an aspect, the iPS cells are obtained from an allogeneic source. In an aspect, the iPS cells are obtained from a syngeneic source. In an aspect, the iPS cells are obtained from a combination of sources.

In yet another aspect, the iPS cells of a disclosed method of inhibiting vascular apoptosis and/or apoptosis-related mechanisms in a subject are obtained from fibroblast cells. In a further aspect, the iPS cells are obtained from H9c2 cells. In an aspect, the fibroblast cells or the H9c2 cells are transfected with a vector comprising a nucleic acid molecule encoding at least one stemness factor. In an aspect, the at least one stemness factor comprises c-myc, oct 3/4, Klf4, nanog, or Sox2, or a combination thereof. For example, in an aspect, the stemness factors are c-myc, oct 3/4, Klf4, and Sox2.

In an aspect, the apoptosis and/or apoptosis-related mechanisms occurs in the induced pluripotent stem cells (iPS cells). In an aspect, the apoptosis and/or apoptosis-related mechanisms occurs in the cardiac tissue of the subject. In an aspect, the apoptosis and/or apoptosis-related mechanisms occurs in both the iPS cells and in the cardiac tissue of the subject. In an aspect, a disclosed method inhibits apoptosis and/or apoptosis-related mechanisms in the iPS cells. In an aspect, a disclosed method inhibits apoptosis and/or apoptosis-related mechanisms in the cardiac tissue of the subject. In an aspect, a disclosed method inhibits apoptosis and/or apoptosis-related mechanisms in both the iPS cells and the cardiac tissue of the subject.

In an aspect, a disclosed method of inhibiting vascular apoptosis and/or apoptosis-related mechanisms comprising administering iPS cells to the subject comprises administering between 5,000 and 500,000 induced pluripotent stem cells (iPS cells). In an aspect, a disclosed method comprises administering approximately 100,000 iPS cells. In an aspect, a disclosed method comprises administering less than 100,000 iPS cells.

In an aspect, a disclosed method comprises administering more than 100,000 iPS cells. In an aspect, iPS cells are administered to the subject in one intramyocardial injection. In an aspect, iPS cells are administered to the subject in two or more intramyocardial injections. In an aspect, iPS cells are administered into a peri-infarct zone of the injured myocardium. In an aspect, iPS cells are administered into an infarcted zone of the injured myocardium. In an aspect, iPS cells are administered into both a peri-infarct zone of the injured myocardium and an infarcted zone of the injured myocardium.

In an aspect, the administration of the conditioned media to the subject is repeated. For example, in an aspect, the conditioned media is administered to the subject prior to and during cardiac dysfunction. In an aspect, the conditioned media is administered to the subject during and following cardiac dysfunction. For example, in an aspect, the conditioned media is administered to the subject prior to and following cardiac dysfunction. In an aspect, the conditioned media is administered to the subject prior to, during, and following cardiac dysfunction.

In an aspect, the administration of the FGF-9 to the subject is repeated. For example, in an aspect, the FGF-9 is administered to the subject prior to and during cardiac dysfunction. In an aspect, the FGF-9 is administered to the subject during and following cardiac dysfunction. For example, in an aspect, the FGF-9 is administered to the subject prior to and following cardiac dysfunction. In an aspect, the FGF-9 is administered to the subject prior to, during, and following cardiac dysfunction.

In an aspect of a disclosed method of inhibiting vascular apoptosis and/or apoptosis-related mechanisms comprising administering FGF-9 to the subject and further comprising administering one or more other components (such as, for example, fibroblast growth factor-9 primed induced pluripotent stem cells, conditioned media of fibroblast growth factor-9 primed induced pluripotent stem cells, and/or a composition comprising fibroblast growth factor-9 and fibroblast growth factor-8), the FGF-9 is administered to the subject prior to the administration of the one or more other components. In an aspect, FGF-9 is administered to the subject during the administration of the one or more other components. In an aspect, FGF-9 is administered to the subject following the administration of the one or more other components. In an aspect, the administration of FGF-9 is repeated. For example, in an aspect, FGF-9 is administered to the subject prior to and during the administration of the one or more other components. In an aspect, FGF-9 is administered to the subject prior to and following the administration of the one or more other components. In an aspect, FGF-9 is administered to the subject during and following the administration of the one or more other components. In an aspect, FGF-9 is administered to the subject prior to, during, and following the administration of the one or more other components.

In an aspect of a disclosed method of inhibiting vascular apoptosis and/or apoptosis-related mechanisms comprising administering the conditioned media of fibroblast growth factor-9 primed induced pluripotent stem cells to the subject and further comprising administering one or more other components (such as, for example, fibroblast growth factor-9 primed induced pluripotent stem cells, fibroblast growth factor-9, and/or a composition comprising fibroblast growth factor-9 and fibroblast growth factor-8), the conditioned media is administered to the subject prior to the administration of the one or more other components. In an aspect, the conditioned media is administered to the subject during the administration of the one or more other components. In an aspect, the conditioned media is administered to the subject following the administration of the one or more other components. In an aspect, the administration of the conditioned media 9 is repeated. For example, in an aspect, the conditioned media is administered to the subject prior to and during the administration of the one or more other components. In an aspect, the conditioned media is administered to the subject prior to and following the administration of the one or more other components. In an aspect, the conditioned media is administered to the subject during and following the administration of the one or more other components. In an aspect, the conditioned media is administered to the subject prior to, during, and following the administration of the one or more other components.

In an aspect, in a disclosed method of inhibiting vascular apoptosis and/or apoptosis-related mechanisms comprising administering to the subject FGF-9, the FGF-9 and/or the one or more other components are administered to the subject within 10, 15, 20, 25, 30, or more minutes following cardiac dysfunction. In an aspect, in a disclosed method of inhibiting vascular apoptosis and/or apoptosis-related mechanisms comprising administering to the subject FGF-9, the FGF-9 and/or the one or more components are administered to the within 1, 2, 6, 12, 18, 24, or more hour following cardiac dysfunction.

In an aspect, in a disclosed method of inhibiting vascular apoptosis and/or apoptosis-related mechanisms comprising administering to the subject the conditioned media, the conditioned media and/or the one or more other components are administered to the subject within 10, 15, 20, 25, 30, or more minutes following cardiac dysfunction. In an aspect, in a disclosed method of inhibiting vascular apoptosis and/or apoptosis-related mechanisms comprising administering to the subject the conditioned media, the conditioned media and/or the one or more components are administered to the within 1, 2, 6, 12, 18, 24, or more hour following cardiac dysfunction.

In an aspect, a disclosed method comprise administering one or more immunosuppressive drugs. Immunosuppressive drugs are known in the art. In an aspect, the one or more immunosuppressive drugs comprise corticosteroids, calcineurin inhibitors, anti-proliferatives, and mTOR inhibitors. In an aspect, the one or more immunosuppressive drugs can be a combination of immunosuppressive drugs. In an aspect, the one or more immunosuppressive drugs is cyclosporine A. In an aspect, the one or more immunosuppressive drugs is administered to the subject prior to, during, and/or following the administration of the induced pluripotent stem cells (iPS cells). In an aspect, the one or more immunosuppressive drugs is administered to the subject prior to the administration of the iPS cells. In an aspect, the one or more immunosuppressive drugs is administered to the subject during the administration of the iPS cells. In an aspect, the one or more immunosuppressive drugs is administered to the subject following the administration of the iPS cells. In an aspect, the administration of the one or more immunosuppressive drugs is repeated. In an aspect, the one or more immunosuppressive drugs is administered to the subject prior to and during the administration of the iPS cells. In an aspect, the one or more immunosuppressive drugs is administered to the subject during and following the administration of the iPS cells. In an aspect, the one or more immunosuppressive drugs is administered to the subject prior to and following the administration of the iPS cells.

In an aspect, a disclosed method for attenuating vascular apoptosis and/or apoptosis-related mechanisms inhibits fibrosis and/or fibrosis-related mechanisms.

In an aspect, a disclosed method for attenuating vascular apoptosis and/or apoptosis-related mechanisms protects myocardium including, but not limited to human myocardium, with or without the administration of iPS cells.

5. Non-Medical Uses

Also disclosed herein are uses of a disclosed composition and methods as investigational and/or research tools in the development and standardization of in vitro and in vivo test systems for evaluation in laboratory animals such as cats, dogs, rabbits, monkeys, rats and mice, as part of the search for new therapeutic approaches for the enhancement of neovascularization as well as for the attenuation of vascular apoptosis and/or apoptosis-related mechanisms. In an aspect, these approaches apply to subjects with cardiac dysfunction.

### C. DEFINITIONS

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the amino acid abbreviations are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

“Peptide” as used herein refers to any peptide, oligopeptide, polypeptide, gene product, expression product, or protein. For example, a peptide can be an enzyme. A peptide is comprised of consecutive amino acids. The term “peptide” encompasses naturally occurring or synthetic molecules.

In general, the biological activity or biological action of a peptide refers to any function exhibited or performed by the peptide that is ascribed to the naturally occurring form of the peptide as measured or observed in vivo (i.e., in the natural physiological environment of the protein) or in vitro (i.e., under laboratory conditions). For example, a biological activity of caspase includes caspase enzymatic activity.

The term “enzyme” as used herein refers to any peptide that catalyzes a chemical reaction of other substances without itself being destroyed or altered upon completion of the reaction. Typically, a peptide having enzymatic activity catalyzes the formation of one or more products from one or more substrates. Such peptides can have any type of enzymatic activity including, without limitation, the enzymatic activity or enzymatic activities associated with enzymes such as those disclosed herein.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or can not occur, and that the description includes instances where said event or circumstance occurs and instances where it does not.

As used herein, the terms “transformation” and “transfection” mean the introduction of a nucleic acid, e.g., an expression vector, into a recipient cell including introduction of a nucleic acid to the chromosomal DNA of said cell. The art is familiar with various compositions, methods, techniques, etc. used to effect the introduction of a nucleic acid into a recipient cell. The art is familiar with such compositions, methods, techniques, etc for both eukaryotic and prokaryotic cells. The art is familiar with such compositions, methods, techniques, etc. for the optimization of the introduction and expression of a nucleic acid into and within a recipient cell.

As used herein, the term “subject” refers to the target of administration, e.g., an animal. Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal A patient refers to a subject afflicted with a disease or disorder. The term “patient” includes human and veterinary subjects. In an aspect of a disclosed method, the subject has been diagnosed with cardiac dysfunction, such as, for example, a cardiac ischemia/reperfusion event, prior to the administering step.

As used herein, the term “treatment” refers to the medical management of a subject or a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In an aspect, the disease, pathological condition, or disorder is cardiac dysfunction, such as, for example, a cardiac ischemia/reperfusion event.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with cardiac dysfunction” means having been subjected to a physical examination by a person of skill, for example, a physician, and found to have a condition that can be diagnosed or treated by a compound or composition that alleviates or ameliorates cardiac dysfunction. As a further example, “diagnosed with a need for “improving or enhancing neovascularization” refers to having been subjected to a physical examination by a person of skill, for example, a physician, and found to have a condition characterized by cardiac dysfunction and/or cardiac impairment and/or cardiac cell death wherein improving or enhancing neovascularization would be beneficial to the subject. Such a diagnosis can be in reference to a disorder, such as myocardial infarction, and the like, as discussed herein.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to cardiac dysfunction or myocardial infarction) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a disclosed compositions or pharmaceutical preparation comprising a disclosed composition to a subject. Such methods are well known to those skilled in the art and include, but are not limited to, intracardiac administration, intramyocardial administration, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, target receptor, or other biological entity together in such a manner that the compound can affect the activity of the target (e.g., receptor, transcription factor, cell, etc.), either directly; i.e., by interacting with the target itself, or indirectly; i.e., by interacting with another molecule, co-factor, factor, or protein on which the activity of the target is dependent.

As used herein, the term “determining” can refer to measuring or ascertaining a quantity or an amount or a change in expression and/or activity level, e.g., of a nucleotide or transcript or polypeptide. For example, determining the amount of a disclosed transcript or polypeptide in a sample as used herein can refer to the steps that the skilled person would take to measure or ascertain some quantifiable value of the transcript or polypeptide in the sample. The art is familiar with the ways to measure an amount of the disclosed nucleotides, transcripts, polypeptides, etc.

As used herein, the term “level” refers to the amount of a target molecule in a sample, e.g., a sample from a subject. The amount of the molecule can be determined by any method known in the art and will depend in part on the nature of the molecule (i.e., gene, mRNA, cDNA, protein, enzyme, etc.). The art is familiar with quantification methods for nucleotides (e.g., genes, cDNA, mRNA, etc) as well as proteins, polypeptides, enzymes, etc.

It is understood that the amount or level of a molecule in a sample need not be determined in absolute terms, but can be determined in relative terms (e.g., when compare to a control or a sham or an untreated sample).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to cardiac dysfunction or myocardial infarction) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the term “neovascularization” is the formation of functional microvascular networks with red blood cell perfusion

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side affects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side affects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts.

By “modulate” is meant to alter, by increase or decrease. As used herein, a “modulator” can mean a composition that can either increase or decrease the expression level or activity level of a gene or gene product such as a peptide. Modulation in expression or activity does not have to be complete. For example, expression or activity can be modulated by about 10%, 20%, 30%, 40%, 50%, 60%, 70%, 80%, 90%, 95%, 99%, 100% or any percentage in between as compared to a control cell wherein the expression or activity of a gene or gene product has not been modulated by a composition.

As used herein, “EC₅₀,” is intended to refer to the concentration or dose of a substance (e.g., a compound or a drug) that is required for 50% enhancement or activation of a biological process, or component of a process, including a protein, subunit, organelle, ribonucleoprotein, etc. EC₅₀ also refers to the concentration or dose of a substance that is required for 50% enhancement or activation in vivo, as further defined elsewhere herein. Alternatively, EC₅₀ can refer to the concentration or dose of compound that provokes a response halfway between the baseline and maximum response. The response can be measured in an in vitro or in vivo system as is convenient and appropriate for the biological response of interest. For example, the response can be measured in vitro using cultured cardiac cells or in an ex vivo organ culture system with isolated cardiac cells, e.g., cardiomyocytes, vascular smooth muscle cells, endothelial cells, etc). Alternatively, the response can be measured in vivo using an appropriate research model such as rodent, including mice and rats. The mouse or rat can be an inbred strain with phenotypic characteristics of interest such as, for example, obesity or diabetes. As appropriate, the response can be measured in a transgenic or knockout mouse or rat wherein a gene or genes has been introduced or knocked-out, as appropriate, to replicate a disease process.

As used herein, “IC₅₀,” is intended to refer to the concentration or dose of a substance (e.g., a compound or a drug) that is required for 50% inhibition or diminution of a biological process, or component of a process, including a protein, subunit, organelle, ribonucleoprotein, etc. IC₅₀ also refers to the concentration or dose of a substance that is required for 50% inhibition or diminution in vivo, as further defined elsewhere herein. Alternatively, IC₅₀ also refers to the half maximal (50%) inhibitory concentration (IC) or inhibitory dose of a substance. The response can be measured in an in vitro or in vivo system as is convenient and appropriate for the biological response of interest. For example, the response can be measured in vitro using cultured cardiac cells or in an ex vivo organ culture system with isolated cardiac cells (e.g., cardiomyocytes, vascular smooth muscle cells, endothelial cells, etc). Alternatively, the response can be measured in vivo using an appropriate research model such as rodent, including mice and rats. The mouse or rat can be an inbred strain with phenotypic characteristics of interest such as, for example, obesity or diabetes. As appropriate, the response can be measured in a transgenic or knockout mouse or rat wherein the a gene or genes has been introduced or knocked-out, as appropriate, to replicate a disease process.

The term “pharmaceutically acceptable” describes a material that is not biologically or otherwise undesirable, i.e., without causing an unacceptable level of undesirable biological effects or interacting in a deleterious manner. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds can not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C—F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### D. EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

1. General Experiments

a. Establishment of iPS Cell Lines and Differentiation of Cardiac Cell Type

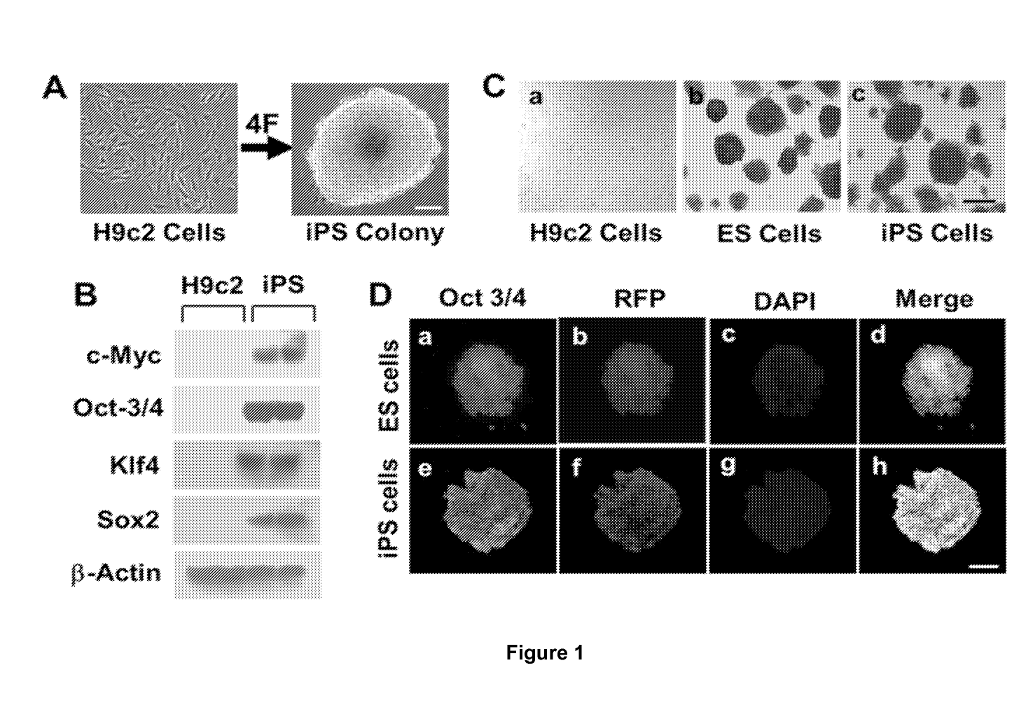

Stably transfected iPS cell lines expressing red fluorescence protein (RFP) were generated. (Park et al., 2008). H9c2 cells were transfected with an expression vector expressing mouse stemness four factors (i.e., the 4F expression vector). The four stemness factors were c-Myc, Oct 3/4, Klf4 and Sox2. FIG. 1 (panel A) shows that untransfected H9c2 cells are elongated (FIG. 1, left panel, scale bar=50 μm). Following transduction with the 4F expression vector, these cells appeared morphologically distinct (FIG. 1A, right panel) from the untransduced H9c2 cells. To determine whether these four factors were present in the generated iPS cells, western blot analysis was utilized to show expression for these four stemness factors in the iPS cells. Western blot analysis did not show the presence of any one of the four factors in the untransfected H9c2 cells, but showed increased expression of 4Fs was present in transduced H9c2. (FIG. 1, panel B). Next, whether generated iPS cells expressed pluripotency markers, such as alkaline phosphatase and Oct3/4, was next determined iPS cells expressed alkaline phosphatase in 100% of growing cells. Similar expression was observed in ES cells, which were used as a positive control (FIG. 1, panel C, scale bar=250 μm). In contrast, H9c2 cells were negative for alkaline phosphatase staining (FIG. 1, panel C). Growing iPS cells were positive for the undifferentiated pluripotent marker Oct3/4, which was compared with the positive control—ES cells (FIG. 1, panel D, scale bar=100 μm). These data indicate that generated iPS cells were reprogrammed and expressed pluripotent markers. (Singla et al., 2011)

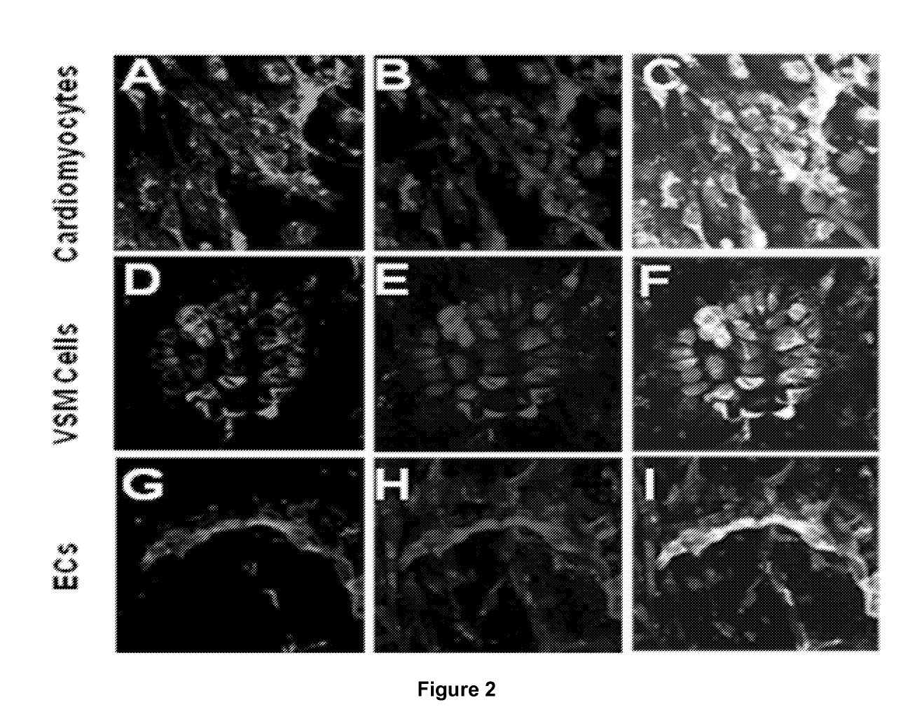

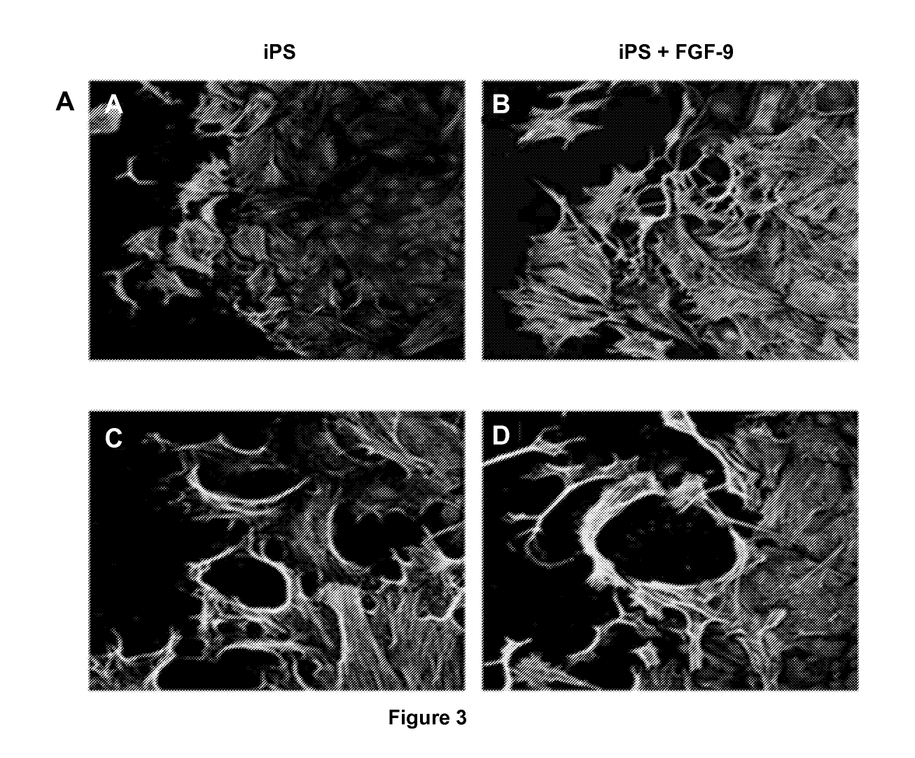

Whether the iPS cell lines retained the capability in vitro to differentiate into all three major heart cell types (i.e., cardiomyocytes, vascular smooth muscle cells and endothelial cells) present and still express the desired reporter protein was evaluated. FIG. 2 shows that iPS cells formed embryoid bodies (EBs) and differentiated into cardiac myocytes, smooth muscle cells and endothelial cells. Here, EBs were differentiated for 17 days. A beating area was stained with anti-myosin/Alexa 488 (panel A), anti-RFP/Alexa 568 (panel B) and DAPI (panel C). The merged image (panel C) shows co-expression of myosin and RFP. Smooth muscle cells were stained with anti-smooth muscle actin/Alexa 488 (panel D), anti-RFP/Alexa 568 (panel E) and DAPI (panel F). The merged image (panel F) shows overlap of expression of smooth muscle α-actin and RFP. Endothelial cells that have formed a capillary-like structure were stained with anti-von Willebrand Factor/Alexa 488 (panel G), anti-RFP/Alexa 568 (panel H) and DAPI (panel I). The merged image (panel I) shows co-expression of von Willebrand Factor and eGFP.

b. FGF-9 Enhancement of EC and VSMC Differentiation from iPS Cells

Normal embryonic cardiogenesis from mesodermal tissue requires growth factors and cytokines, including fibroblast growth factors (FGFs), wnt proteins, bone morphogenic proteins (BMPs), and transforming growth factor (TGF) β (Singla et al., 2005). Several cytokines have been shown to favor cell specific differentiation in mouse ES cells. IL-3 promotes differentiation to macrophages, mast cells, or neutrophils (Wiles et al., 1991), IL-6 to erythroid lineages (Biesecker et al., 1993), retinoic acid to neurons (Slager et al., 1993), and TGFβ to cardiac myocytes (Singla et al., 2005). Recombinant mouse insulin growth factor (IGF-1) was added to a suspension of undifferentiated mouse ES cells and subsequently transplanted into mouse hearts (Kofidis et al., 2005). This study demonstrated restoration of infarcted areas (increased donor graft/infarct ratio) with increased connexin-43 expression (Kofidis et al., 2005).

Similarly, FGF family members play an important role in various cell signal transduction pathways associated with embryo development (Dyer et al., 2009; Yun et al., 2010). Reports on other FGF isoforms (FGF2, FGF4, and FGF-9) indicate a potential role in angiogenesis Yun et al., 2010; Frontini et al., 2011) in the heart whereas FGF-8 has been reported to be pro-cardiac myocyte growth factor in the developing heart (Dyer et al., 2009; Marques et al., 2008). Specifically, FGF-9 recently been reported to decrease mortality in mice following MI (Korf-Klingebiel et al., 2011). However, the role of FGF-9 or iPS cells in the injured diabetic adult heart has not yet been characterized. Therefore, whether iPS cells primed with FGF-9 increase neovascularization and regenerate the injured diabetic myocardium merits further investigation.

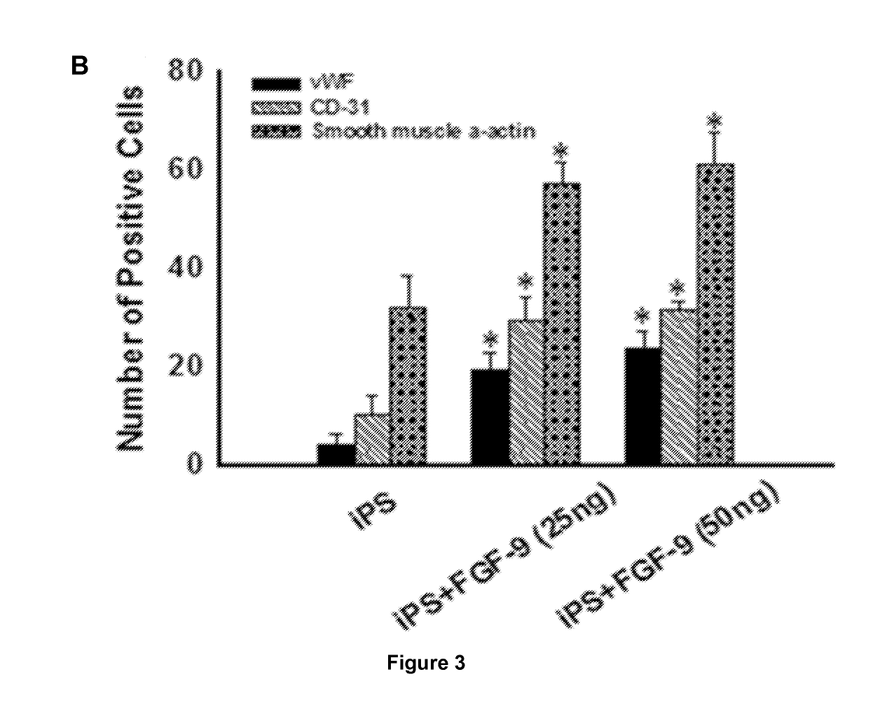

The iPS-EB system was used to determine whether direct treatment with FGF-9 would promote EC and VSMC differentiation. EBs derived from iPS cells were treated with 0, 25, and 50 ng per mL of the bio-active form of recombinant mouse FGF-9 and were examined by microscopy from day 0 (DO) through day 14 (D14) after plating. FIG. 3A shows that FGF-9-treated EBs stained positive for CD31 specific for ECs, smooth muscle α-actin for VSMCs, anti-RFP for iPS cells, and DAPI for nuclear staining (panel A-B). Additionally, the bottom panel of FIG. 3 shows positive staining for von Willebrand factor VIII for ECs. FIG. 3B shows a histogram providing a quantitative analysis of ECs and VSMCs, demonstrating a significant increase in the number of positively stained cells at D14 following FGF-9 treatment.

c. Identification of Factors Released from iPS-CM

Whether cytokine/growth factors were present in the iPS-CMs and could provide protection from H₂O₂-induced apoptosis was examined 69 cytokine/growth factors were analyzed with the use of Luminex technology. (Singla et al., 2008; Singla et al., 2009). Cytoprotective proteins released into iPS-CM were present at higher levels as compared with the H9c2-CM, ES-CM, or cell culture media used as controls. Notably, FGF-9 was significantly increased in the iPS-CM as were FGF-8, IL-10, and TIMP-1.

TABLE 6
IDENTIFICATION OF FACTORS RELEASED FROM IPS-CM

| | | H9c2 | ES | | | |
|---|---|---|---|---|---|---|
| Released | Control | Cells- | Cells- | iPS- | FGF-8- | |
| Factors | # | CM | CM | CM | iPS-CM | Comments |
| FGF-9 | L* | 140^{$} | 140 | 370 | 370 | Pro-cardiac growth |
| | | | | | | factor in embryonic |
| | | | | | | heart |
| IL-10 | L | L | L | 45 | 84 | Anti-apoptotic |
| | | | | | | factor in cardiac |
| | | | | | | myocytes |
| TIMP-1 | 7 | 19 | 130 | 380 | 380 | Anti-apoptotic; also |
| | | | | | | well known as anti- |
| | | | | | | fibrotic |

| | |
|---|---|
| FGF-8 | Pro-cardiac growth factor in the embryonic heart; |
| | its anti-apoptotic and pro-cardiac characteristics |
| | in apoptotic cells and in iPS-EB cell culture system |
| | established herein |

^{$}Values are expressed as pg/mL
*L = lowest detectable dose by the assay
# Cell culture medium used to grow ES cells

d. Establishment of the Myocardial Infarction Model and Cardiac Neovascular Regeneration

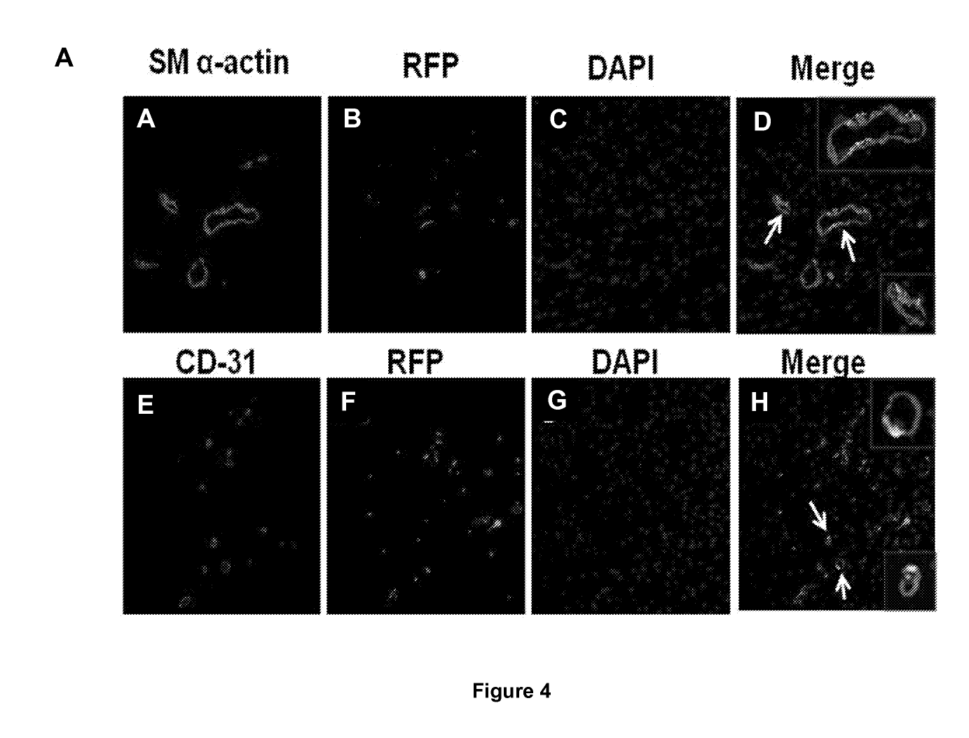

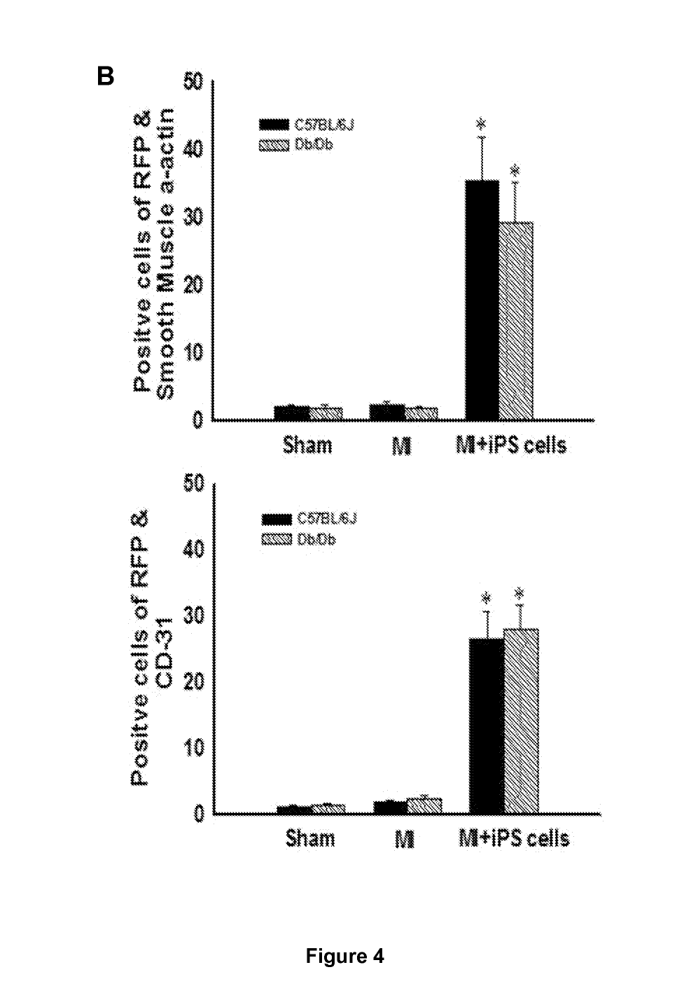

A previously characterized mouse MI model (Singla et al., 2006; Singla et al., 2007; Singla et al., 2001) was used to study iPS cell transplantation. MI was produced in C57BL/6 and db/db mice by left coronary artery ligation Immediately after coronary ligation, two intramyocardial injections of 10 μL of medium±2.5×10⁴ iPS cells were delivered into peri-infarct zone areas of the LV. (Singla et al., 2006; Singla et al., 2007; Singla et al., 2001). iPS cells were injected in the LV of MI animals in both groups and compared with controls (FIG. 4).

The fate of donor cells was determined with the use of co-immunolabeling for the donor cell marker and neovascular cell type-specific proteins. For example, an anti-smooth muscle anti-actin antibody was used to identify VSMCs, and an anti-CD31 antibody was used to identify ECs. In iPS cell transplanted hearts, anti-RFP immunolabeling demonstrated areas of donor cell engraftment in the infarct and border zones. Co-immunolabeling with an antibody specific for VSMCs and ECs revealed small and large artery regeneration. (FIG. 4).

In FIG. 4, transplanted iPS cells following MI demonstrated neovascularization (panels A-D show vascular smooth muscle cells and panels E-H show endothelial cells). Panel A shows smooth muscle cells stained with anti-smooth muscle α-actin/Alexa568, and panel E shows endothelial cells stained with anti-CD31/Alexa568. Panels B and F show donor-derived cells stained with anti-RFP/Alexa 488 (in green) and panels C and G show nuclei stained with DAPI. The co-localization of cell type-specific markers and donor cell RFP in merged images (yellow) indicated that transplanted iPS cells became vascular smooth muscle (panel D), and endothelial cells (panel H). The arrows in panels D and H indicate enlarged areas in right hand corner. In the histogram shown in FIG. 4B, the quantitative data indicate that there was a significant (p<0.05) increase in the number of positively stained VSMCs (top panel) and ECs (bottom panel) in the MI+iPS group as compared with MI hearts.

e. Effects of Transplanted iPS Cells and FGF-9 on C-Kit^{+ve}-CPCs

MI was produced in C57BL/6 and db/db mice by left coronary artery ligation. Cells were transplanted as described above whereas FGF-9 (1 ng/20 μL in two injections) was intramyocardially injected immediately after MI in two different injections in the peri-infarct zone. After D14, we examined c-kit^{+ve} cardiac progenitor cells (CPCs), CD31^{+ve} ECs, SMα-actin and vascular cell apoptosis. Evidence of activated c-kit^{+ve} cells and their co-staining with mature VSMC marker SM α-actin was identified. These findings indicated that transplanted iPS cell released factors or that FGF-9 activated c-kit^{+ve} cells, which then differentiated into mature VSMCs.

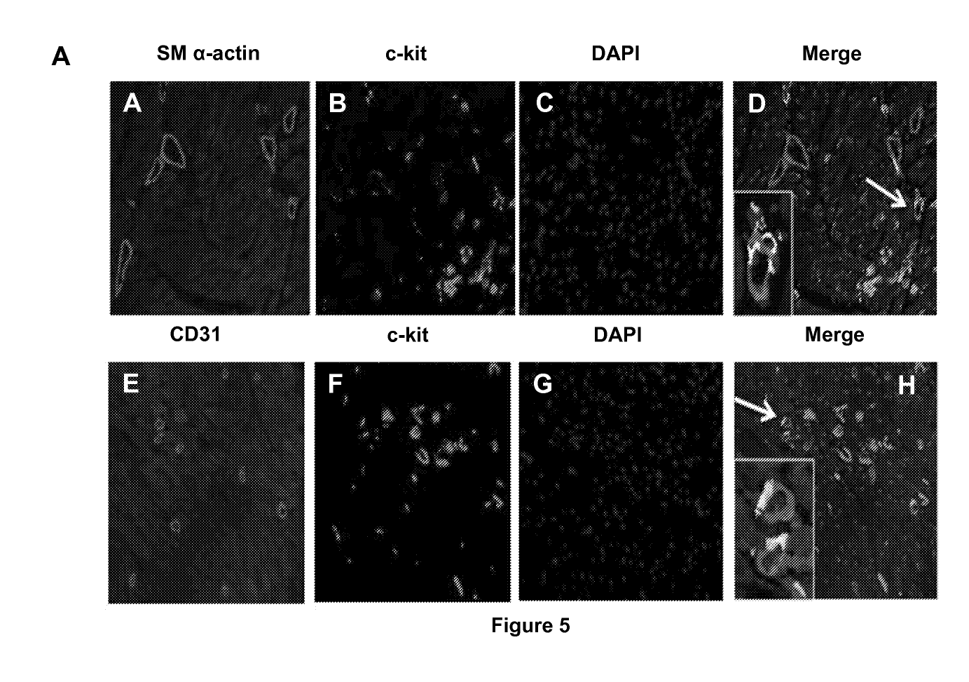

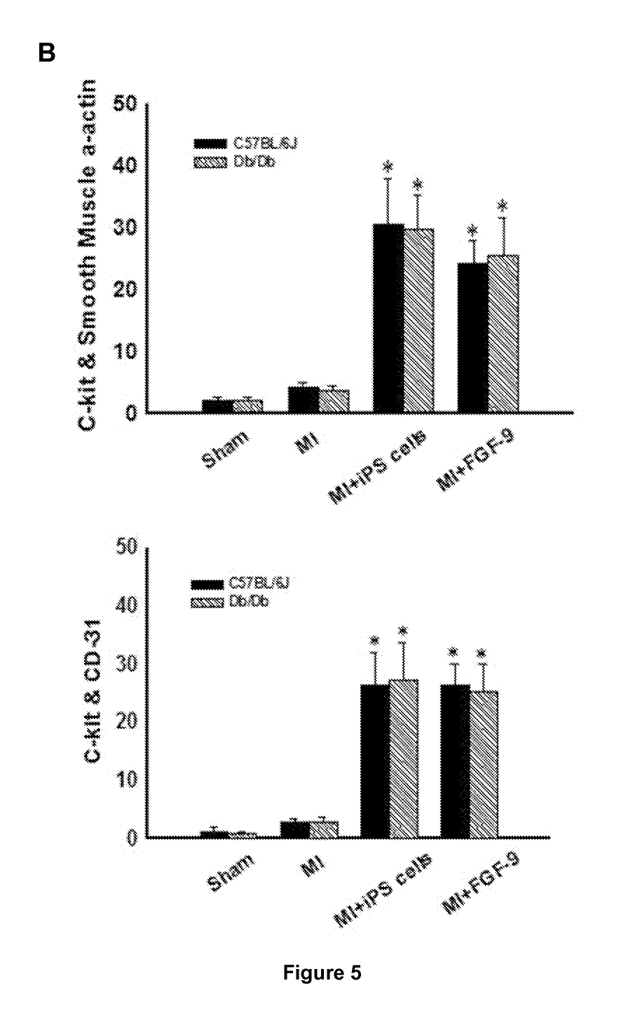

In FIG. 5, panels A-D show c-kit positive cell activation in neovascularization in vascular smooth muscle and panels E-H show activated c-kit^{+ve} cells and co-staining with the mature EC marker CD31. Cell type-specific immunolabeling is indicated in red for smooth muscle (panel A) with anti-smooth muscle α-actin/Alexa568, and for endothelial cells with anti-CD31/Alexa568 (panel E). Activated c-kit positive cells were identified by anti-c-kit/Alexa 488 in green (panels B and F), and nuclei were stained with DAPI in blue (panels C and G). Co-localization of cell type-specific markers were merged images (yellow) indicating that activated c-kit positive cells have become VSMCs (panel D) and ECs (panel H). The arrows in panels D and H indicate enlarged areas. FIG. 5B shows a histogram presenting a quantitative analysis of positive c-kit+VSMCs (top panel) and c-kit+ECs (bottom panel). This quantitative data shows a significantly (*p<0.05) increased number of c-kit^{+ve}+VSMCs and c-kit^{+ve}+ECs in the MI+iPS and MI+FGF-9 groups as compared with MI. Moreover, a cytokine growth analysis that iPS cells secrete pro-angiogenic growth factor FGF-9 was confirmed.

f. Effect of FGF-9 on Caspase Activity and Cell Death

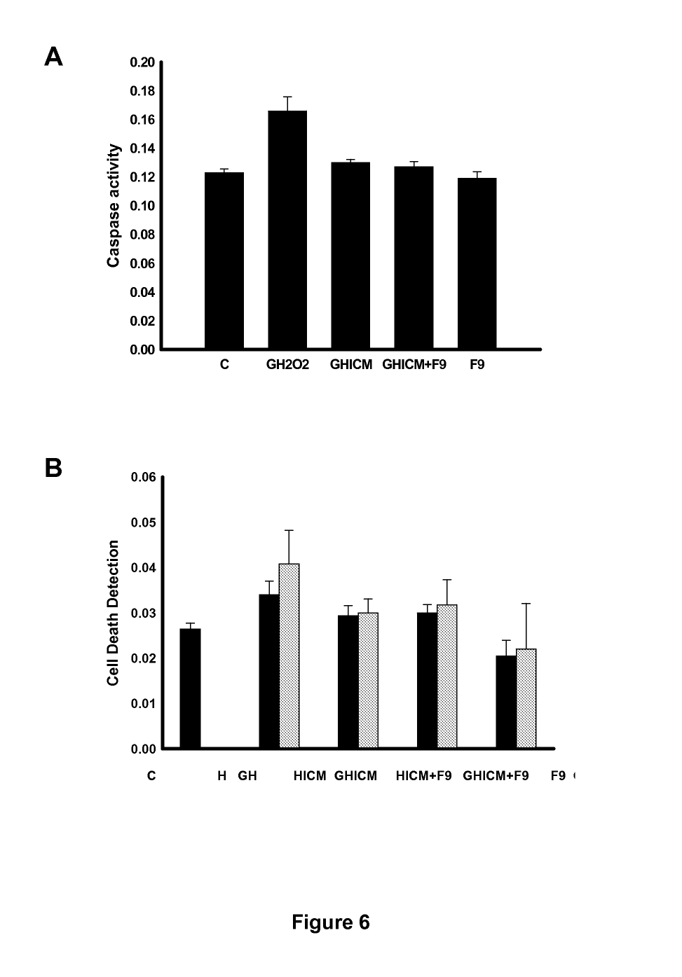

FIG. 6A shows caspase activity following treatment with glucose and H₂O₂ (GH2O2), or glucose and iPS cell conditioned media (GHICM), or glucose and iPS cell conditioned media and FGF-9 (GHICM+F9), or FGF-9 (F9). Control is represented by C.

FIG. 6B shows cell death detection following treatment with treatment with H₂O₂ (H), or glucose and H₂O₂ (GH), or glucose and H₂O₂ (GH) and iPS cell conditioned media (GHICM), or H₂O₂ and iPS cell conditioned media and FGF-9 (HICM+F9), or glucose and H₂O₂ and iPS cell conditioned media and FGF-9 (GHICM+F9), or FGF-9 (F9). Control is represented by C. In FIG. 6B, black bars indicates that H9c2 cells were treated with H₂O₂ to induce cell death while gray bars indicated that glucose was provided to the H₂O₂ treated cells.

g. Formation of Vessels Following iPS Cell and FGF-9 Transplantation

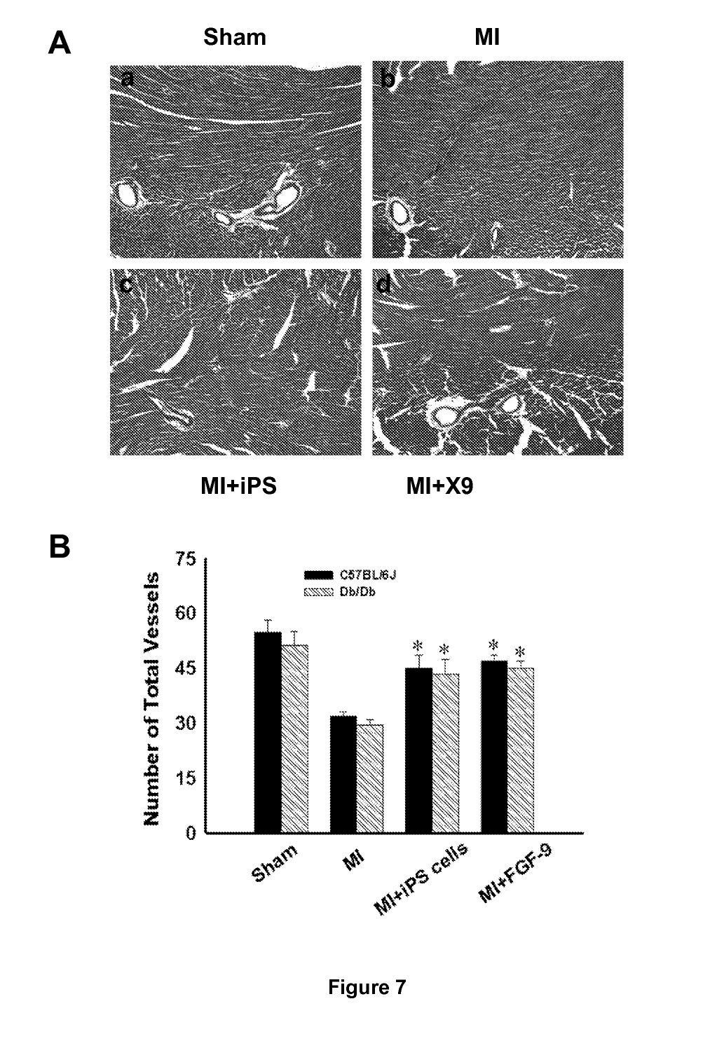

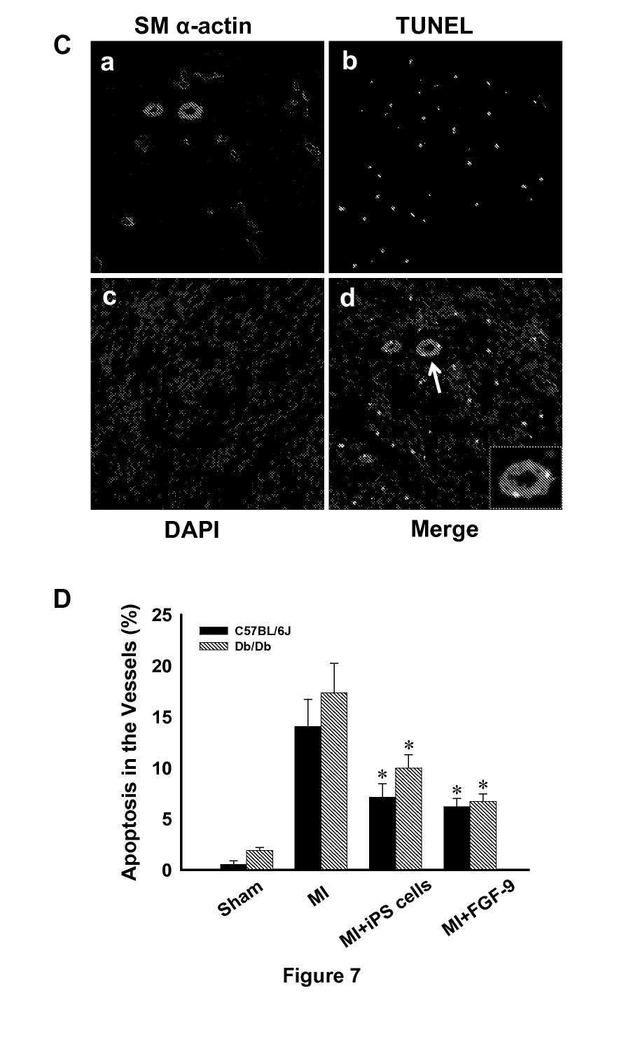

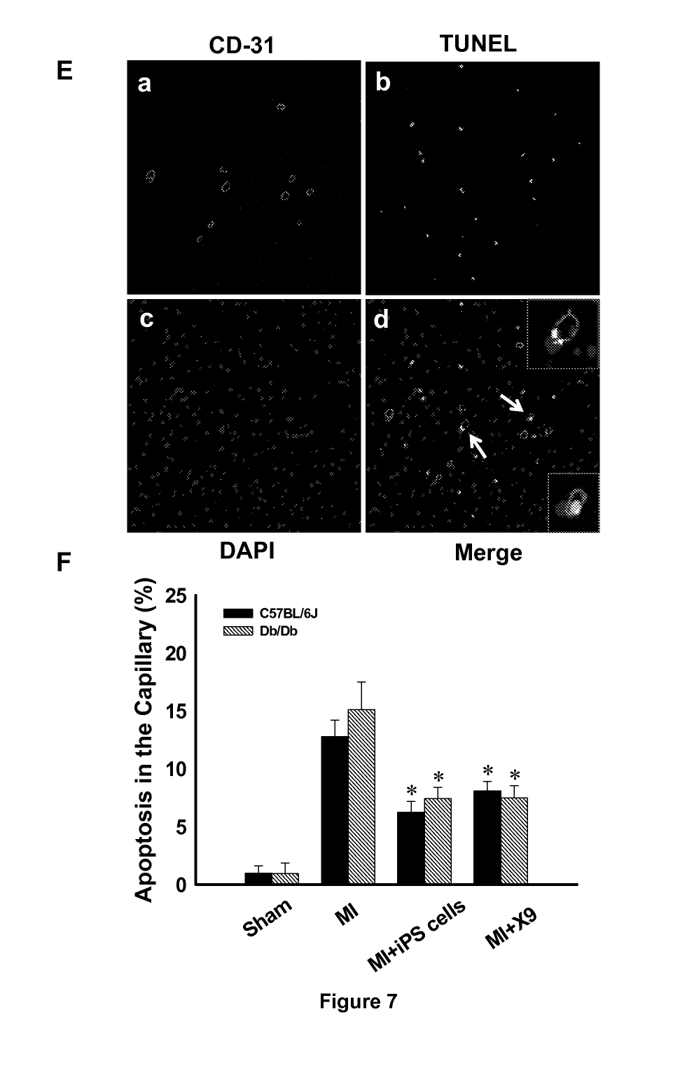

To determine the effect of transplanted iPS cells and FGF-9 on coronary artery formation, the total number of small, medium, and large vessels was counted using H&E and Masson's trichome staining (See, e.g., FIG. 7A showing Masson staining in db/db mice following sham-MI (panel a), MI (panel b), MI+iPS cell treatment (panel c), and MI+FGF-9 treatment (panel d). The total number of vessels in the transplanted iPS cell and FGF-9 groups were compared with MI in both C57BL/6J and db/db mice following MI. The histogram in FIG. 7B shows a significant increase in the number of vessels in the MI+iPS cells group and the MI+FGF-9 group as compared to the MI group (* p<0.05). An increase in vessel formation following iPS cell or FGF-9 transplantation was confirmed. (X9=FGF-9).

FIG. 7C shows staining for smooth muscle (anti-alpha-actin antibody) in C57BL/6J mice following MI (panel a). FIG. 7C also shows TUNEL staining in panel b and DAPI staining in panel c. The merged images of shown in panel d. The histogram in FIG. 7D shows a significant decrease in the % of apoptosis in the vessels in the both the C57BL/6J and db/db mice following MI and treatment with either iPS cells or FGF-9 (* p<0.05 compared to the MI group).

Moreover, FIG. 7E shows staining for endothelial cells (anti-CD31 antibody) in C57BL/6J mice following MI (panel a). FIG. 7E also shows TUNEL staining in panel b and DAPI staining in panel c. The merged images of shown in panel d. The histogram in FIG. 7F shows a significant decrease in the % of apoptosis in the capillaries in the both the C57BL/6J and db/db mice following MI and treatment with either iPS cells or FGF-9 (* p<0.05 compared to the MI group).

h. Effects of Treatment with iPS Cells and iPS Cells with FGF-9 on Cell Differentiation

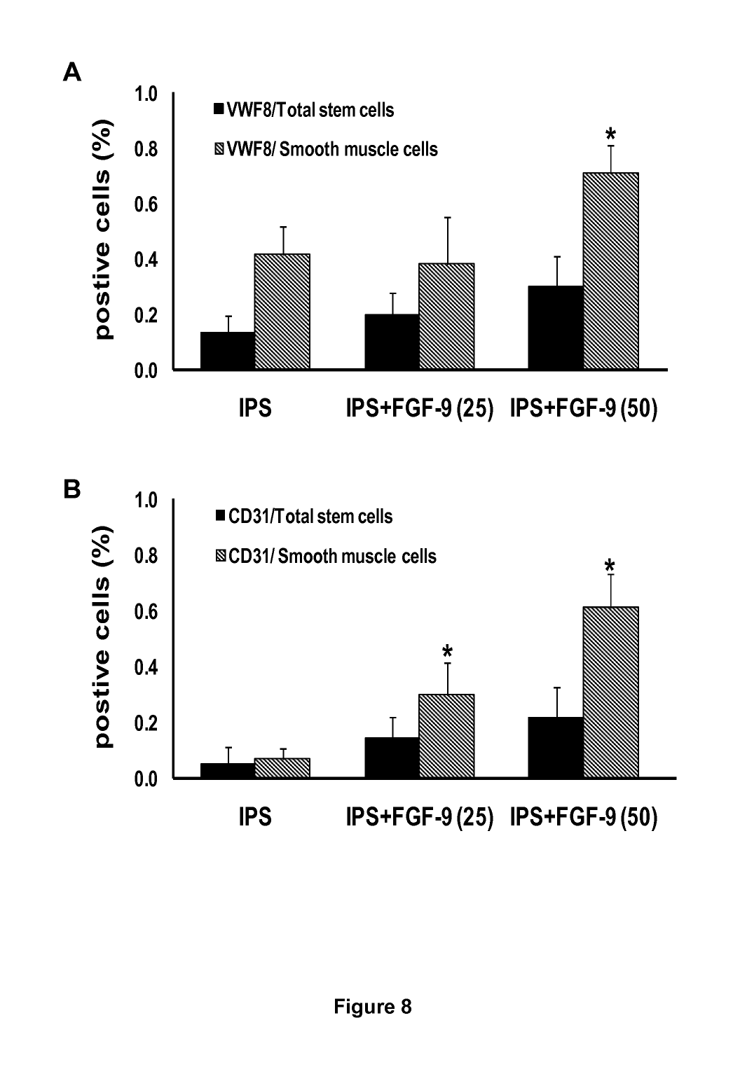

FIG. 8A shows the percentage of total stem cells positively stained for vWF8 and the total number of smooth muscle cells positively stained for vWF8 in the iPS cell-treated group, the iPS cell-treated group with FGF-9 (25 nanograms), and the iPS-cell treated group with FGF-9 (50 nanograms). FIG. 8b shows the percentage of total stem cells positively stained for CD21 and the total number of smooth muscle cells positively stained for CD21 in the iPS cell-treated group, the iPS cell-treated group with FGF-9 (25 nanograms), and the iPS-cell treated group with FGF-9 (50 nanograms).

i. Effects of FGF-9 on MIR-126 Expression

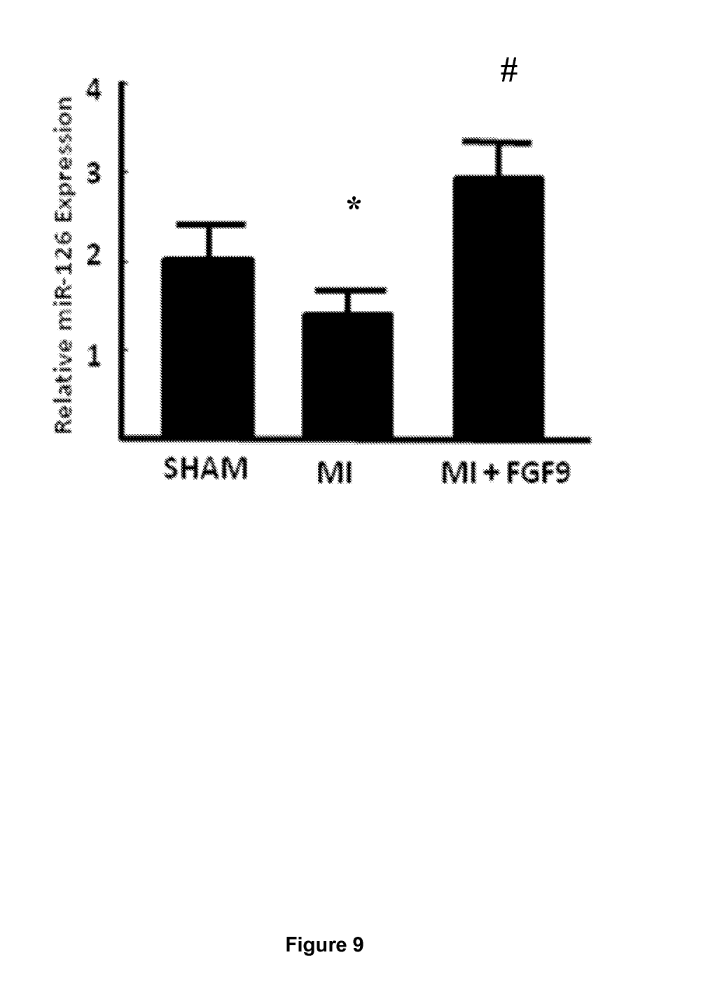

Following MI, there is a significant decrease in cardiac neovascularization. The data presented herein show that this decrease is blunted following FGF-9 treatment. Furthermore, the data presented herein indicate that levels of miR-126 expression significantly decreased following MI and this decrease was modulated following FGF-9 treatment. The histogram in FIG. 9 shows a decrease in miR-126 levels in the MI group as compared to sham control. This decrease in the MI group was reversed following FGF-9 treatment. (*p<0.05 v. sham and #p<0.05 v. MI). These data indicate that miR-126 is involved in FGF-9 mediated neovascularization.

j. Echocardiographic Evidence of Improvement in LV Function

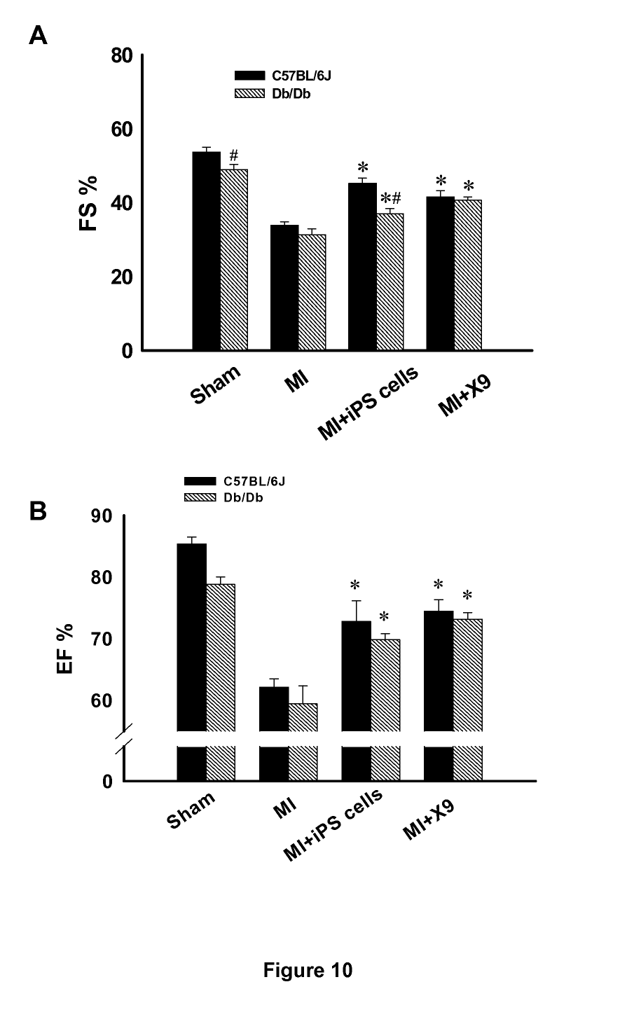

M-mode echocardiography was used as previously reported to determine the effect of iPS cells and FGF-9 transplantation on LV size and function in C57BL/6 and db/db mice 2 weeks following MI. FIG. 10 shows that iPS cell and FGF-9 treatment blunted post-MI remodeling and improved cardiac function two weeks following MI. FIG. 10A shows the fractional shortening (FS) for the iPS cells or FGF-9 treatment groups were significantly different from the MI group. (*p<0.05). FIG. 10B shows the ES for the iPS cells or FGF-9 treatment groups were significantly different from the MI group. (*p<0.05). (X9=FGF-9).

k. Avoidance of Teratoma Formation

Both human and mouse ES cells form complex teratomas when engrafted into an immune deficient host, which is a characteristic of ES cells resulting from their pluripotential capacity (Damjanov 2004; Thomson et al., 1998). Teratoma formation has been considered a major potential limitation in the therapeutic use of ES cells especially when the transplanted undifferentiated number of cells is higher than 100,000 (Nussbaum et al., 2007). A recent study suggests that intramyocardial transplantation of 1×10⁶ ES or iPS (generated from fibroblasts) cells in the mouse heart causes teratomas (Nelson et al., 2009; Nussbaum et al., 2007). In contrast, no teratoma formation was observed with transplantation of 300,000 cells (Bhfar et al., 2002).

The transplantation of 3×10⁴ undifferentiated ES cells did not result in the formation of teratomas after 2 (Singla et al., 2006) and 4 weeks. Importantly, generated iPS cells from various cell sources might have varied in their epigenetic alterations; therefore formation of teratomas may vary. No teratoma formation was observed in the infarcted heart following H9c2 cell-induced iPS cell transplantation (5×10⁴ undifferentiated iPS cells) (Singla et al., 2011). Noticeably, the number of transplanted iPS cells was dramatically less compared with the studies reporting teratoma formation (i.e., range varies from 100,000 to one million undifferentiated ES or iPS cells) (Nussbaum et al., 2007).

l. Examination of Immunogenicity of iPS Cells

iPS cells from various sources may vary in their epigenetic alterations (Kim et al., 2010). One study suggested that iPS cells generated from fibroblasts using retroviral and episomal approaches are immunogenic in nature (Zhao et al., 2011). However, generated iPS cells by the episomal approach were less immunogenic compared with the cells generated by the retroviral approach (Zhao et al., 2011). iPS cells generated from H9c2 cells using plasmid transfection were used herein, but the immunogenicity of these cells is not well understood. Importantly, the method, the number of cells, and the location of injections described herein differ from recent reports on the immunogenicity of iPS cells (Zhao et al., 2011). Moreover, ES cells transplanted in the sheep heart were compared with and without the immunosuppressant drug cyclosporine A, suggesting there was engraftment and cardiac regeneration in both groups (Menard et al., 2005).

2. Determination of Whether Transplanted FGF-9 Primed iPS Cells Attenuate Vascular Cell Apoptosis and Enhance Neovascular Repair and Regeneration Following MI

Diabetes mellitus (Type II) combined with MI leads to severe cardiac and vascular remodeling, which is a complex, dynamic, and time-dependent process (Glass et al., 2010; Abel 2005; Amos et al., 1997). Recently, neovascularization has been achieved in MI with transplanted endothelial progenitor cells (EPCs) isolated from peripheral blood as well as adult and ES cell sources (Jujo et al., 2008; Tillmanns et al., 2008; Urbanket et al., 2003). Mobilization of EPCs in pigs, mouse, and rat models of MI demonstrate an increase in capillary density and improved heart function (Jujo et al., 2008; Tillmanns et al., 2008; Urbanket et al., 2003). Next, experimental evidence indicate that in diabetic animals increased blood glucose levels inducing oxidative stress lead to cardiac and vascular apoptosis, hypertrophy, and vascular remodeling (Abel 2005; Urbanek 2005). Angiogenesis abnormalities have been shown to be present in the infarcted non-diabetic and diabetic heart (Fatma et al., 2010; Abel 2005; Urbanek et al., 2005) How much neovascularization can be achieved in the infarcted non-diabetic and diabetic hearts following transplantation of iPS cells primed with pro-angiogeneic factor FGF-9 is unknown

(1) Determination of the Effects of FGF-9 Primed iPS Cells on Neovascular Regeneration in Non-Diabetic and Diabetic Mice Following MI

These experiments assess the effects of FGF-9 primed iPS cells following transplantation in infarcted myocardium (non-diabetic and diabetic mice) on stem cell survival, engraftment, and differentiation into neovascular cell types. An established mouse myocardial infarction (MI) model (Singla et al., 2006; Singla et al., 2007; Singla et al., 2011) to inject RFP labeled differentiated iPS cells primed with FGF-9 (total of 1×10⁵ cells in two different intramyocardial injections in the peri-infarct zone of the infarcted heart. For example, differentiated iPS, ES cells, fibroblast reprogrammed iPS cells, H9c2 cells, FGF-9 (1 ng/20 μL in two injections or 50 ng/mL, or cell culture medium (as a control) are injected. The subsequent effects on neovascularization are quantified.

C57BL/6 and db/db (Jackson Laboratories) mice are used and the mice are separated into groups receiving or not receiving Cyclosporin A. Animals are treated with cyclosporine A (250-300 mg/kg) for 5 days before cell transplantation and are treated with cyclosporine A for 28 days after cell transplantation (see, e.g., Menard et al., 2005; Giralt et al., 1997). Drug dose are increased or decreased based on average cyclosporine serum concentration of 300-400 ng/L (Menard et al., 2005; Giralt et al., 1997). Moreover, before and after MI, db/db mice are regularly monitored for glucose and given insulin injection (0.75 U/kg i.p. as reported (Kobayashi et al., 2008) if glucose levels exceed 350±25 mg/dL to eliminate the variation of observed differences due to glucose levels. Animals are killed humanely at short-term (72 hrs and 14 days) and long-term (3, 6, 12, and 16 weeks) for histological, immunohistochemistry, and physiological (blood flow) analysis.

FGF-9 primed-iPS cells are treated as follows. FGF-9 (50 ng/mL) is added to the iPS cells in the differentiation medium (a medium that does not contain iPS cell self-renewal growth factors such as activin A, leukemia inhibitory factor and mouse embryonic fibroblast condition medium) for 48 hours. These iPS cells are considered as FGF-9 primed differentiated iPS cells. Control iPS or ES cells are cultured for 48 hrs in a differentiation medium without FGF-9. Infiltration of T-cells is determined using anti-CD3 and anti-CD4 antibodies (Zhao et al., 2011). H&E stained sections are examined to identify the presence of any inflammatory cells. Additionally, heart sections are analyzed to determine the effect of FGF-9-iPS cells on capillary density and formation of coronary arteries. Heart function is examined via echocardiography (Singla et al., 2006; Singla et al., 2011)

(2) Determination of Total Number of Transplanted FGF-9 Primed iPS Cells Engraftment Following MI

Serial sections containing right ventricular (RV), LV free wall, and interventricular septum are immunostained for RFP (Evrogen, Russia), Oct3/4, or Sox2 (Santa Cruz Biotechnology) to determine early engraftment up to 24 hrs. Cells that stain positive for RFP, but not for Oct3/4 or Sox2 antibody, are considered as non-engrafted iPS cells. Sections are counter-stained with DAPI (Sigma) to delineate the nuclei. Fluorescence microscopy is used to acquire images. NIH image J processing and analysis software are employed for these analyses.

(3) Determination of Proliferation of Engrafted Cell Proliferation Following MI

To determine the number of engrafted iPS cells that have started EC and VSMC proliferation, BrdU (50 mg/kg body weight, i.p (Beltrami et al., 2003) is administered. BrdU identifies nuclei in S phase in order to label actively dividing cells. Active cell growth of engrafted cells is identified using double-label immunostaining for RFP and BrdU (Dako) antibodies. RFP identifies donor engrafted cells whereas BrdU labels DNA synthesizing cells. Furthermore, to determine how many cells have started cell division and cell types at any given time, double-label immunostaining is performed using a Ki-67 antibody (Dako) and an RFP antibody. Ki-67 is a nuclear antigen, which is associated with cell division and labels proliferating cells (G1-, S-, G2-phase and mitosis), but not in quiescent or resting cells (G0-phase), and as stated above, RFP antibodies identify donor grafted cells. Slides are analyzed using fluorescence (Olympus) and confocal microscopy.

Cell stage differentiation is determined Using double or triple-label immunostaining to detect donor cells (RFP antibody) and early, late, and mature differentiated EC cell types using their marker cell specific antibodies, the number of early and mature endothelial cells that form following engraftment at various time points following MI is assessed. If cells stain positive with CD14⁺ or CD1a⁺ antibodies and are negative for vWF antibody staining, then these cells are considered early EPCs. If cells stain positively for CD34⁺ or CD133⁺ antibodies and are negative for vWF antibody staining, then these cells are considered late EPCs Mature endothelial cells are identified with antibodies such as factor VIII antibody (Sigma), CD31/PECAM-1 (Santa Cruz), and Griffonia simplicifolia lectin (Sigma).

Reports state that EPCs have the potential to differentiate into ECs and VSMCs. To confirm that EPCs differentiated from transplanted iPS cells are also positive with both newly formed EC and VSM progenitor cells, sections are immunostained with endothelial and smooth muscle specific markers (e.g., FLK-1/VEGF-R2, CD34, VEcad, and endoglin). Sections are stained with respective FITC or rhodamine-conjugated secondary antibodies and counterstained with DAPI for nuclear visualization. Slides are analyzed using Olympus and confocal microscopy. Western blotting and RT-PCR are be used to determine the SM cell markers such as SM-MHC-11, calponin and SM22 alpha. The early and late EPC marker antibodies are commercially available (Krenning et al., 2008).

(4) Identification of Coronary Artery Formation Following MI

New cell differentiation and capillary and coronary artery formation are determined using double-label immunostaining to detect donor cells (RFP antibody) and differentiated cell types using neovascular cell specific antibodies. Endothelial cells are identified using cell specific antibodies such as factor VIII antibody (Sigma), CD31/PECAM-1 (Santa Cruz), and Griffonia simplicifolia lectin (sigma). For the identification of smooth muscle cells, α-smooth muscle (SM) actin (Sigma), SM22a, calponin, and SM-myosin heavy chain (MHC)-11 antibodies are used. Sections are stained with respective FITC or rhodamine conjugated secondary antibodies and counterstained with DAPI for nuclear visualization. Slides are analyzed using confocal microscopy.

Whether newly formed vessels are functional, and whether the vessels survive long term (or whether the vessels have atrophied or been eliminated due to apoptosis) are examined Immunostaining is performed and the size and number of vessels is determined To distinguish the extent of cell survival versus apoptotic cell death at long term time points, newly formed coronary arteries are identified using a combination of RFP staining, cell specific staining, and TUNEL and caspase-3/Bax antibody. Sections are stained with respective FITC or rhodamine conjugated secondary antibodies and counterstained with DAPI (15 μg/mL for nuclear visualization. Slides are analyzed using Olympus fluorescence and confocal microscopy. Furthermore, animals are treated with rhodamine-labeled dextran to determine whether newly generated coronary vessels are functional and have blood circulation. (Fatma et al., 2010; Singla et al., 2006; Singla et al., 2008; Singla et al., 2011 (Mol. Pharm); Singla et al., 2011 (Am. J. Physiol. Heart Cir. Physiol.).

(5) Determination of Regional Blood Flow Following MI

To determine if transplanted stem cells give rise to increased blood flow through new vessel formation, the commonly used non-radioactive isotope-labeled microspheres procedure is performed as described in Tillmans et al., 2008).

(6) Functional Assessment of the Regenerated Heart

Echocardiography is an accurate noninvasive tool for the determination of quantitative characterization of heart remodeling following MI (Singla et al., 2008; Singla et al., 2011 (Am. J. Physiol. Heart Circ. Physiol.). Echocardiography is performed on infarcted hearts at short term post-MI time points (72 hrs and 14 days) and at long-term post-MI time points (3, 6, 12, and 16 weeks). M-mode images in a short axis view are performed to measure LV mass, LV mass to body ratio, LV anterior and posterior wall thickness, and fractional shortening.

3. Determination of Whether FGF-9-iPS-CM Enhances Endogenous Activation of C-Kit⁺ Flk-1-ve CPCs and FLK1^{+ve}-PCs Following MI

Factors released from Akt transfected mesenchymal stem cells inhibit cardiac myocyte apoptosis (Gnecchi et al., 2005; Gnecchi et al., 2006). Some studies indicate that MSCs following transplantation in the infarcted heart restores c-kit positive CSCs niches (Hare 2007) and inhibit apoptosis. CPCs proliferation and differentiation was also activated by intramyocardial injection of HGF and IGF-1 in the infarcted myocardium (Tillmanns et al., 2008; Urbanek et al., 2003). Factors released from ES cells are different than those factors released from adult stem cells and inhibit H9C2 cell apoptosis (Singla et al., 2007; Singla et al., 2008). Following transplantation of ES-CM in the infarcted heart, cardiac myocyte apoptosis was significantly inhibited and function was improved. (Fatma et al., 2010; Singla et al., 2011 (Am J Physiol Heart Circ Physiol). Following transplantation of ES-CM in the infarcted heart, there was enhanced activation of CPCs and FLK-1^{+ve}-PCs. (Fatma et al., 2010; Singla et al., 2011). The enhanced activation contributed to cardiac neovascular regeneration via improved cardiac function. Moreover, following the intramyocardial injection of FGF-9 in the infarcted heart, there was enhanced neovascularization mediated via c-kit positive cells.

(1) Determination of the Effects of Factors Released from iPS Cells on C-Kit^{+ve} CPCs and Flk-1^{+ve}-PC Following Myocardial Infarction

Using a cell culture model system (Fatma et al., 2010; Singla et al., 2007; Singla et al., 2008; Singla et al., 2011), iPS-CM is produced from iPS-cells primed with and without FGF-9. Preparation of CM is well reported by us. A mouse myocardial infarction (MI) model (Fatma et al., 2010; Singla et al., 2007; Singla et al., 2008; Singla et al., 2011) is used as well as C57BL/6 and db/db mice with and without cyclosporine A to inject FGF-9-iPS-CM (15× (Fatma et al., 2010; Singla et al., 2011), FGF-9 (1 ng/20 μL in two injections (50 ng/mL) or cell culture medium (as a control) using two intramyocardial injections following MI. Moreover, additional cell specific CM such ES-CM and H9c2-CM are used for comparison purposes. The effects on cardiac neovascularization are quantified. Animals are killed humanely at short time points (72 hrs and 14 days) and long-term time points (3, 6, 12 and 16 weeks) following MI treatment for histological, physiological analysis, and preparation of tissue homogenates. To analyze the effect of iPS-CM, heart sections are prepared for immunostaining to determine CPCs and FLK-1-PCs niches and cell proliferation and differentiation. Echocardiography is used to examine heart function

(2) Identification of C-Kit^{-ve} CPCs and Flk-1^{+ve}-PCs and their Proliferation and Cell Division

Following transplantation in the infarcted heart, resident CPCs have been identified and the role of these CPCs in neovascular regeneration has been determined. CPCs are lineage negative and are identified with cell specific markers. CPCs specific antibodies such as c-kit, MDR1 and sca-1 (Molecular Probes) are used to identify CPCs. To distinguish whether CPCs are migrated from the bone marrow, sections are stained for markers of hematopoietic cell lineage (such as CD34, Cd8, and CD45 (Molecular Probes, Santa Cruz)). FLK-1 specific antibodies such as CD34 and FLK-1/VEGF-R2 (Santa Cruz) are used to identify FLK-1 cells. The number of CPCs and FLK-1-PCs that have started proliferation and differentiation is determined.

(3) Identification of Progenitor Neovascular Cell Types Lineage

Using double-label immunostaining to detect CPCs (c-kit, MDR1 and sca-1 antibody) and using cell specific antibodies to detect neovascular specific progenitor cell types such as VSMCs and ECs using (Fatma et al., 2010), cell specific lineage progenitor cells are determined To determine progenitor ECs, sections are immunostained with endothelial specific transcription factors Ets-1, Fli-1, Etv2, and Erg 1 (Sigma, Molecular Probes) and structural protein flk1 (Santa Cruz) antibodies. For progenitor smooth muscle cells, transcription factors GATA 6 and GATA 2 and structural protein α-SM actin antibodies (Sigma) are used. Sections will then be stained with respective FITC or rhodamine conjugated secondary antibodies and counterstained with DAPI for nuclear visualization. Slides are analyzed using Olympus fluorescence and confocal microscopy. The number of CPCs in the acute and chronic cardiomyopathy is quantified. RT-PCR and western blot is performed to confirm increased levels of endothelial and vascular smooth muscle cells transcriptional factors and protein levels.

4. Determination of Whether Transplanted FGF-9 Primed iPS Cells or FGF-9-iPS-CM Stimulates Neovascularization Via Activation of MIR-126 Pathway

miRNAs are small non-coding RNAs about 20-24 nucleotides in length that play a major role in the regulation of a variety of cell processes including apoptosis, hypertrophy, fibrosis, and cardiac differentiation (Care et al., 2007; Catalucci et al., 2008; Chen et al., 2006; Cimmino et al., 2005). The deletion of miR-126 was reported to demonstrate loss in vascular integrity and defects in ECs proliferation in the mice with and without myocardial infarction (Wang et al., 2008). Furthermore, miR-126 inhibited SPRED1 and PIK3R2, which are both negative regulators of the VEGF signaling pathway (Wang et al., 2008; Heusschen et al., 2010).

These studies determine the role of the miR-126 pathway in neovascularization in diabetic infarcted mouse hearts as well as determine the effect of transplanted FGF-9 primed iPS cells or FGF-9-iPS-CM. The presence of decreased neovascularization that leads to decreased cardiac function in the infarcted mouse heart was demonstrated. Next, transplanted iPS cells enhanced neovascularization with improved cardiac function. Moreover, the levels of miR-126 were significantly reduced following MI and this decrease was reversed with cell transplantation.

(1) Determination of the Effects of FGF-9-iPS Cells or iPS-CM on MIR-126 Mediated Neovascularization

Using RT-PCR, the levels of miR-126 following MI are determined (Glass et al., 2011) and miR-126 levels are confirmed with northern blotting (Dey et al, 2011; Narducci et al., 2011; Small et al., 2010). To determine the role of miR-126 on neovascularization, animals are injected with a selective inhibitor of miR-126 (locked nucleic acid, LNA-antagomir-126, 80 mg/kg, as this dose is used by other investigators (Hartley et al., 2010; Patrick et al., 2010). To identify expression of miR-126 in the infarcted, non-infarcted, and peri-infarcted region of the heart following MI, heart sections are stained using in situ-hybridization for miR-126. In situ-hybridization employing LNA-miR-126 specific probes (Exiqon) (Hosada et al., 2011) is utilized. Additionally, expression of miR-126 in major heart cell types is characterized using double label immunostaining with cell specific antibodies. In brief, after in situ hybridization, sections are co-labeled with neovascular cell specific antibodies such as CD31 for endothelial cells and smooth muscle α-actin for VSM cells. Sections are counter stained with DAPI to identify cell nuclei.

To determine the effects of miR-126 on downstream targeted protein SPREAD1 and PIK3R2, SDS-PAGE and Western blot analyses are performed to determine the protein expression of total and phosphorylated SPRED1 and PIK3R2 (Abcam, USA). Densitometry is used to measure band density.

All of the compositions and/or methods disclosed and claimed herein can be made and executed without undue experimentation in light of the present disclosure. It will be apparent to those skilled in the art that various modifications and variations can be made in the present invention without departing from the scope or spirit of the invention.

More specifically, certain agents which are both chemically and physiologically related can be substituted for the agents described herein while the same or similar results can be achieved. All such similar substitutes and modifications apparent to those skilled in the art are deemed to be within the spirit, scope and concept of the invention as defined by the appended claims.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### E. REFERENCES

The following references, to the extent that they provide exemplary procedural or other details supplementary to those set forth herein, are specifically incorporated herein by reference.
Abel, E. D. Myocardial insulin resistance and cardiac complications of diabetes. Curr. Drug Targets. Immune. Endocr. Metabol. Disord. 5, 219-226 (2005).
Amos, A. F., et al. The rising global burden of diabetes and its complications: estimates and projections to the year 2010. Diabet. Med. 14 Suppl 5, S1-85 (1997).
Behfar, A. et al. Stem cell differentiation requires a paracrine pathway in the heart. FASEB J. 16, 1558-1566 (2002).
Behr, B., et al. FGF-9 is required for angiogenesis and osteogenesis in long bone repair. Proc. Natl. Acad. Sci. U.S.A 107, 11853-11858 (2010).
Beltrami, A. P. et al. Adult cardiac stem cells are multipotent and support myocardial regeneration. Cell 114, 763-776 (2003).
Biesecker, L. G. et al. Interleukin-6 is a component of human umbilical cord serum and stimulates hematopoiesis in embryonic stem cells in vitro. Exp. Hematol. 21, 774-778 (1993).
Care, A. et al. MicroRNA-133 controls cardiac hypertrophy. Nat. Med. 13, 613-618 (2007).
Catalucci, D., Latronico, M. V., & Condorelli, G. MicroRNAs control gene expression: importance for cardiac development and pathophysiology Ann. N. Y. Acad. Sci. 1123, 20-29 (2008).
Chang, M. Y. et al. Direct reprogramming of rat neural precursor cells and fibroblasts into pluripotent stem cells. PLoS. One. 5, e9838 (2010).
Chen, J. F. et al. The role of microRNA-1 and microRNA-133 in skeletal muscle proliferation and differentiation. Nat. Genet. 38, 228-233 (2006).
Cimmino, A. et al. miR-15 and miR-16 induce apoptosis by targeting BCL2. Proc. Natl. Acad. Sci. U.S.A 102, 13944-13949 (2005).
Damjanov, I. From stem cells to germ cell tumors and back. Verh. Dtsch. Ges. Pathol. 88, 39-44 (2004).
Dey, B. K., et al. miR-206 and -486 induce myoblast differentiation by downregulating Pax7. Mol. Cell Biol. 31, 203-214 (2011).
Dyer, L. A. et al. The role of secondary heart field in cardiac development. Dev. Biol. 336, 137-144 (2009).
Fatma, S., et al. Factors Released from Embryonic Stem Cells Stimulate c-kit-FLK-1(+ve) Progenitor Cells and Enhance Neovascularization. Antioxid. Redox. Signal. 13, 1857-1865 (2010).
Frontini, M. J. et al. Fibroblast growth factor 9 delivery during angiogenesis produces durable, vasoresponsive microvessels wrapped by smooth muscle cells. Nat. Biotechnol. 29, 421-427 (2011).
Giralt, S. et al. Phase I trial of cyclosporine-induced autologous graft-versus-host disease in patients with multiple myeloma undergoing high-dose chemotherapy with autologous stem-cell rescue. J. Clin. Oncol. 15, 667-673 (1997).
Glass, C. E., et al. Stem cells in the diabetic infarcted heart. Heart Fail. Rev. (2010).
Gnecchi, M. et al. Evidence supporting paracrine hypothesis for Akt-modified mesenchymal stem cell-mediated cardiac protection and functional improvement. FASEB J. 20, 661-669 (2006).
Gnecchi, M. et al. Paracrine action accounts for marked protection of ischemic heart by Akt-modified mesenchymal stem cells. Nat. Med. 11, 367-368 (2005).
Hatley, M. E. et al. Modulation of K-Ras-dependent lung tumorigenesis by MicroRNA-21. Cancer Cell 18, 282-293 (2010).
Heusschen, R., et al. MicroRNAs in the tumor endothelium: novel controls on the angioregulatory switchboard. Biochim. Biophys. Acta 1805, 87-96 (2010).
Hosoda, T. et al. Human cardiac stem cell differentiation is regulated by a mircrine mechanism. Circulation 123, 1287-1296 (2011).
Jujo, K., et al. Endothelial progenitor cells in neovascularization of infarcted myocardium. J. Mol. Cell Cardiol. 45, 530-544 (2008).
Kim, K. et al. Epigenetic memory in induced pluripotent stem cells. Nature 467, 285-290 (2010).
Kobayashi, M. et al. Expression of Src homology 2 domain-containing protein tyrosine phosphatase substrate-1 in pancreatic beta-Cells and its role in promotion of insulin secretion and protection against diabetes. Endocrinology 149, 5662-5669 (2008).
Kofidis, T. et al. Stimulation of paracrine pathways with growth factors enhances embryonic stem cell engraftment and host-specific differentiation in the heart after ischemic myocardial injury. Circulation 111, 2486-2493 (2005).
Korf-Klingebiel, M. et al. Conditional transgenic expression of fibroblast growth factor 9 in the adult mouse heart reduces heart failure mortality after myocardial infarction. Circulation 123, 504-514 (2011).
Krenning, G., et al. Generating new blood flow: integrating developmental biology and tissue engineering. Trends Cardiovasc. Med. 18, 312-323 (2008).
Kuzmenkin, A. et al. Functional characterization of cardiomyocytes derived from murine induced pluripotent stem cells in vitro. FASEB J. 23, 4168-4180 (2009).
Lunde, K. et al. Intracoronary injection of mononuclear bone marrow cells in acute myocardial infarction. N. Engl. J. Med. 355, 1199-1209 (2006).
Marques, S. R., et al. Reiterative roles for FGF signaling in the establishment of size and proportion of the zebrafish heart. Dev. Biol. 321, 397-406 (2008).
Menard, C. et al. Transplantation of cardiac-committed mouse embryonic stem cells to infarcted sheep myocardium: a preclinical study. Lancet 366, 1005-1012 (2005).
Narducci, M. G. et al. MicroRNA profiling reveals that miR-21, miR486 and miR-214 are upregulated and involved in cell survival in Sezary syndrome. Cell Death. Dis. 2, e151 (2011).
Nelson, T. J. et al. Repair of acute myocardial infarction by human stemness factors induced pluripotent stem cells. Circulation 120, 408-416 (2009).
Nussbaum, J. et al. Transplantation of undifferentiated murine embryonic stem cells in the heart: teratoma formation and immune response. FASEB J. 21, 1345-1357 (2007).
Park, I. H. et al. Disease-specific induced pluripotent stem cells. Cell 134, 877-886 (2008).
Park, I. H., et al. Generation of human-induced pluripotent stem cells. Nat. Protoc. 3, 1180-1186 (2008).
Patrick, D. M. et al. Stress-dependent cardiac remodeling occurs in the absence of microRNA-21 in mice. J. Clin. Invest 120, 3912-3916 (2010).
Singla, D. K., et al. Factors released from embryonic stem cells inhibit apoptosis of H9c2 cells. Am. J. Physiol Heart Circ. Physiol 293, H1590-H1595 (2007).
Singla, D. K., et al. Enhancement by growth factors of cardiac myocyte differentiation from embryonic stem cells: A promising foundation for cardiac regeneration. Biochem. Biophys. Res. Commun 335, 637-642 (2005).
Singla, D. K. Embryonic stem cells in cardiac repair and regeneration. Antioxid. Redox. Signal. 11, 1857-1863 (2009).
Singla, D. K. et al. Transplantation of embryonic stem cells into the infarcted mouse heart: formation of multiple cell types. J. Mol. Cell Cardiol. 40, 195-200 (2006).
Singla, D. K. Stem cells in the infarcted heart. J. Cardiovasc. Transl. Res. 3, 73-78 (2010).
Singla, D. K., et al. iPS Cells Repair and Regenerate Infarcted Myocardium. Mol. Pharm. (2011).
Singla, D. K., et al Transplanted embryonic stem cells following mouse myocardial infarction inhibit apoptosis and cardiac remodeling. Am. J. Physiol Heart Circ. Physiol 293, H1308-H1314 (2007).
Singla, D. K., et al. Factors released from embryonic stem cells inhibit apoptosis in H9c2 cells through PI3K/Akt but not ERK pathway. Am. J. Physiol Heart Circ. Physiol 295, H907-H913 (2008).
Singla, D. K., et al TGF-beta2 treatment enhances cytoprotective factors released from embryonic stem cells and inhibits apoptosis in infarcted myocardium. Am. J. Physiol Heart Circ. Physiol 300, H1442-H1450 (2011).
Slager, H. G., et al. Transforming growth factor-beta in the early mouse embryo: implications for the regulation of muscle formation and implantation. Dev. Genet. 14, 212-224 (1993).
Small, E. M. et al. Regulation of PI3-kinase/Akt signaling by muscle-enriched microRNA-486. Proc. Natl. Acad. Sci. U.S.A 107, 4218-4223 (2010).
Takahashi, K., et al. Induction of pluripotent stem cells from mouse embryonic and adult fibroblast cultures by defined factors. Cell 126, 663-676 (2006).
Tendera, M. et al. Intracoronary infusion of bone marrow-derived selected CD34+CXCR4+ cells and non-selected mononuclear cells in patients with acute STEMI and reduced left ventricular ejection fraction: results of randomized, multicentre Myocardial Regeneration by Intracoronary Infusion of Selected Population of Stem Cells in Acute Myocardial Infarction (REGENT) Trial Eur. Heart J. 30, 1313-1321 (2009).
Thomson, J. A. et al. Embryonic stem cell lines derived from human blastocysts. Science 282, 1145-1147 (1998).
Tillmanns, J. et al. Formation of large coronary arteries by cardiac progenitor cells. Proc. Natl. Acad. Sci. U.S.A 105, 1668-1673 (2008).
Urbanek, K. et al. Intense myocyte formation from cardiac stem cells in human cardiac hypertrophy. Proc. Natl. Acad. Sci. U.S.A 100, 10440-10445 (2003).
Urbanek, K. et al. Myocardial regeneration by activation of multipotent cardiac stem cells in ischemic heart failure. Proc. Natl. Acad. Sci. U.S.A 102, 8692-8697 (2005).
Wang, S. et al. The endothelial-specific microRNA miR-126 governs vascular integrity and angiogenesis. Dev. Cell 15, 261-271 (2008).
White, A. C., et al. FGF-9 and SHH regulate mesenchymal Vegfa expression and development of the pulmonary capillary network. Development 134, 3743-3752 (2007).
Wiles, M. V., et al. Multiple hematopoietic lineages develop from embryonic stem (ES) cells in culture. Development 111, 259-267 (1991).
Yun, Y. R. et al. Fibroblast growth factors: biology, function, and application for tissue regeneration. J. Tissue Eng 2010, 218142 (2010).
Zhang, J. et al. Functional cardiomyocytes derived from human induced pluripotent stem cells. Circ. Res. 104, e30-e41 (2009).
Zhao, T., et al. Immunogenicity of induced pluripotent stem cells. Nature (2011).

1. A method of enhancing neovascularization following cardiac dysfunction in a subject in need thereof, the method comprising:
administering to the subject (i) fibroblast growth factor-9 primed induced pluripotent stem cells; (ii) conditioned medium of fibroblast growth factor-9 primed induced pluripotent stem cells; (iii) fibroblast growth factor-9; (iv) a composition comprising fibroblast growth factor-9 and fibroblast growth factor-8; or (vi) a combination thereof, and
determining neovascularization in the heart.

2. A method of attenuating vascular apoptosis or apoptosis-related mechanisms following cardiac dysfunction in a subject in need thereof comprising:
administering to the subject (i) fibroblast growth factor-9 primed induced pluripotent stem cells; (ii) conditioned medium of fibroblast growth factor-9 primed induced pluripotent stem cells; (iii) fibroblast growth factor-9; (iv) a composition comprising fibroblast growth factor-9 and fibroblast growth factor-8; or (v) a combination thereof, and
determining apoptosis or apoptosis related mechanisms in the heart.

3. (canceled)

4. The method of claim 1, wherein enhancing neovascularization comprises enhancing cell engraftment, enhancing cell proliferation, enhancing cell differentiation, or a combination thereof.

5. (canceled)

6. (canceled)

7. (canceled)

8. (canceled)

9. The method of claim 1, further comprising enhancing cardiac function in the subject.

10. The method of claim 8, wherein enhancing cardiac function comprises increasing cardiac blood flow, increasing cardiac capillary density, or a combination thereof.

11. (canceled)

12. The method of claim 1, wherein the conditioned media comprises anti-apoptotic and anti-fibrotic factors.

13. The method of claim 12, wherein anti-apoptotic factors and anti-fibrotic comprise fibroblast growth factor-8, fibroblast growth factor-9, interleukin-10, and tissue inhibitor of matrix metalloproteinase-1.

14. The method of claim 1, further comprising attenuating vascular apoptosis or apoptosis-related mechanisms.

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. The method of claim 1, further comprising administering to the subject one or more immunosuppressive drugs.

27. The method of claim 26, wherein the one or more immunosuppressive drugs comprise corticosteroids, calcineurin inhibitors, anti-proliferatives, and mTOR inhibitors.

28. The method of claim 26, wherein the one or more immunosuppressive drugs is cyclosporine A.

29. (canceled)

30. (canceled)

31. The method of claim 30, wherein administering the fibroblast growth factor-9 to the subject occurs prior to, during, and/or following the administration of the induced pluripotent stem cells.

32. (canceled)

33. (canceled)

34. (canceled)

35. (canceled)

36. (canceled)

37. (canceled)

38. The method of claim 1, wherein administering the induced pluripotent stem cells to the subject occurs prior to, during, and/or following cardiac dysfunction.

39. (canceled)

40. (canceled)

41. (canceled)

42. (canceled)

43. (canceled)

44. (canceled)

45. (canceled)

46. A composition for enhancing neovascularization or attenuating vascular apoptosis or apoptosis-related mechanisms in a subject, the composition comprising (i) fibroblast growth factor-9 primed induced pluripotent stem cells; (ii) conditioned medium of fibroblast growth factor-9 primed induced pluripotent stem cells; (iii) fibroblast growth factor-9; (iv) fibroblast growth factor-9 and fibroblast growth factor-8, or (v) a combination thereof.

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. The composition of claim 46, wherein the conditioned media comprises anti-apoptotic and anti-fibrotic factors.

64. The composition of claim 63, wherein anti-apoptotic factors and anti-fibrotic comprise fibroblast growth factor-8, fibroblast growth factor-9, interleukin-10, and tissue inhibitor of matrix metalloproteinase-1.

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. The composition of claim 46, further comprising one or more immunosuppressive drugs.

78. The composition of claim 77, wherein the one or more immunosuppressive drugs comprise corticosteroids, calcineurin inhibitors, anti-proliferatives, and mTOR inhibitors, or wherein the one or more immunosuppressive drugs comprises cyclosporine A.

79. (canceled)

80. (canceled)

81. (canceled)

82. The composition of claim 46, wherein the induced pluripotent stem cells are obtained from fibroblast cells or from H9c2 cells.

83. (canceled)

84. The composition of claim 82, wherein the fibroblast cells or the H9c2 cells are transfected with a vector comprising a nucleic acid molecule encoding at least one stemness factor, and wherein the at least one stemness factor comprises c-myc, oct 3/4, Klf4, nanog, Sox2, and/or a combination thereof.

85. (canceled)

86. (canceled)

87. (canceled)

88. (canceled)
